(19) United States
(12) Patent Application Publication
(10) Pub. No.: US 2022/0370267 A1
(43) Pub. Date: Nov. 24, 2022

(54) ABSORBENT ARTICLE

(71) Applicant: Kimberly-Clark Worldwide, Inc., Neenah, WI (US)

(72) Inventors: Mayara C. Carvalho, Suzano (BR); Ricardo Abarca, Suzano (BR); Rogerio B. Bastos, Sao Paulo (BR)

(21) Appl. No.: 17/753,982

(22) PCT Filed: Nov. 6, 2019
(86) PCT No.: PCT/US19/59974

§ 371 Date: Mar. 21, 2022

(51) Int. Cl. A61F 13/511; A61F 13/475

(52) U.S. Cl. CPC A61F 13/51108; A61F 13/4756; A61F 2013/530007

(57) ABSTRACT

An absorbent article can have a topsheet layer, a backsheet layer, and an absorbent core positioned between the topsheet layer and the backsheet layer. The absorbent core can have a primary absorbent region having a first height measured from the topsheet layer facing surface to the backsheet layer facing surface and a first density, a secondary absorbent region having a second height measured from the topsheet layer facing surface to the backsheet layer facing surface and a second density, and a tertiary absorbent region having a third height measured from the topsheet layer facing surface to the backsheet layer facing surface and a third density. The first height is greater than the second height and the second height is greater than the third height. The first density is the same as the second density. The third density is greater than each of the first density and second density.

### BACKGROUND OF THE DISCLOSURE

Products such as absorbent articles are often used to collect and retain human body exudates containing, for example, urine, menses, and/or blood. Comfort, absorbency, and discretion are three main product attributes and areas of concern for the wearer of the absorbent article. In particular, a wearer is often interested in knowing that such products will absorb significant volumes of body exudates with minimal leakage in order to protect their undergarments, outer garments, or bedsheets from staining, and that such products will help them avoid the subsequent embarrassment brought on by such staining.

Currently, a wide variety of products for absorption of body exudates are available in the form of feminine pads, sanitary napkins, panty shields, and pantiliners. These products generally have an absorbent core positioned between a body-facing liquid permeable topsheet layer and a garment-facing liquid impermeable backsheet layer. The edges of the topsheet layer and the backsheet layer are often bonded together at their periphery to form a seal to contain the absorbent core and body exudates received into the product through the topsheet layer. In use, products such as, for example, feminine pads and sanitary napkins are typically positioned in the crotch portion of an undergarment for absorption of the body exudates and a garment attachment adhesive on the backsheet layer can be used to attach the product to the inner crotch portion of the undergarment. Some of these products can also include wing-like structures for wrapping about the wearer's undergarment to further secure the product to the undergarment and to protect the undergarment from staining. Such wing-like structures (also known as flaps or tabs) are frequently made from lateral extensions of the topsheet and/or backsheet layers.

Wearers of such absorbent articles, however, desire discretion, comfort, and close to the body fit. Conventional absorbent articles which are placed in the wearer's undergarment may not be able to provide such desired benefits to the wearer. Absorbent articles which are attached to a wearer's undergarment may experience twisting, contorting, and shifting out of place as they are subjected to the movement of the wearer's undergarment. Such twisting, contorting, and shifting of the absorbent article can result in the formation of a gap between the body of the wearer of the absorbent article and the absorbent article itself. The presence of the gap can be a cause of concern to the wearer of the absorbent article due to the lack of a close to the body fit. The presence of the gap can result in a reduction in the performance of the absorbent article to capture and absorb body exudates directly into the absorbent article.

As a result, there remains a need for an absorbent article which is comfortable to wear, has an improved ability to capture and absorb body exudates, and inhibits leakage from the absorbent article.

### SUMMARY OF THE DISCLOSURE

In various embodiments, an absorbent article can have a topsheet layer; a backsheet layer; an absorbent core positioned between the topsheet layer and the backsheet layer, the absorbent core can have a topsheet layer facing surface and a backsheet layer facing surface; a first primary absorbent region having a first height measured from the topsheet layer facing surface to the backsheet layer facing surface and a first density; a secondary absorbent region having a second height measured from the topsheet layer facing surface to the backsheet layer facing surface and a second density; and a tertiary absorbent region having a third height measured from the topsheet layer facing surface to the backsheet layer facing surface and a third density, wherein a portion of the tertiary absorbent region is adjacent to a portion of the primary absorbent region; wherein the first height is greater than the second height, the second height is greater than the third height, the first density is the same as the second density, and the third density is greater than each of the first density and the second density.

In various embodiments, the tertiary absorbent region is formed by an embossment.

In various embodiments, a portion of the tertiary absorbent region extends in a longitudinal direction of the absorbent article. In various embodiments, a portion of the tertiary absorbent region is in an overlapping configuration with a longitudinal axis of the absorbent article.

In various embodiments, the first primary absorbent region crosses over a longitudinal axis of the absorbent article.

In various embodiments, the absorbent article can further have a second primary absorbent region having a fourth height measured from the topsheet layer facing surface to the backsheet layer facing surface and a fourth density wherein the fourth height is the same as the first height and the fourth density is the same as each of the first density and the second density. In various embodiments, a portion of the tertiary absorbent region is located between the first primary absorbent region and the second primary absorbent region of the absorbent article. In various embodiments, the tertiary absorbent region is formed by an embossment. In various embodiments, a portion of the tertiary absorbent region extends in a longitudinal direction of the absorbent article. In various embodiments, a portion of the tertiary absorbent region is in an overlapping configuration with a longitudinal axis of the absorbent article. In various embodiments, neither of the first primary absorbent region or the second primary absorbent region cross over a longitudinal axis of the absorbent article.

In various embodiments, the absorbent core comprises a cellulosic fluff material. In various embodiments, the absorbent core further has a superabsorbent material.

In various embodiments, the absorbent article further has a distribution layer. In various embodiments, the distribution layer is positioned between the absorbent core and the backsheet layer of the absorbent article.

### BRIEF DESCRIPTION OF DRAWINGS

FIG. 1 is a top down view of an exemplary embodiment of an absorbent article.

FIG. 2A is an exemplary embodiment of a cross-sectional view of the absorbent article of FIG. 1 taken along line 2A-2A.

FIG. 2B is an exemplary embodiment of a cross-sectional view of the absorbent article of FIG. 1 taken along line 2B-2B.

FIG. 3 is a schematic side elevation of an apparatus for forming an airlaid absorbent core.

FIG. 4 is a schematic perspective of a drum of the apparatus of FIG. 3.

FIG. 5 is a fragmentary cross-section of the apparatus of FIG. 3.

FIG. 6 is a bottom perspective of a form member of the apparatus of FIG. 3.

FIG. 7 is a top plan view of the form member of the apparatus of FIG. 3.

FIG. 8 is a fragmentary section taken in the plane including line 8-8 of FIG. 4.

FIG. 9 is a cross-section of an embodiment of a scarfed absorbent core formed by the apparatus of FIG. 3.

FIG. 10 is a cross-section of an embodiment of a scarfed absorbent core formed by the apparatus of FIG. 3.

FIG. 11A is an exemplary embodiment of a cross-section view of the absorbent article of FIG. 1 taken along line 11A-11A.

FIG. 11B is an exemplary embodiment of a cross-sectional view of the absorbent article of FIG. 1 taken along line 11B-11B.

FIG. 12 is a top down view of an exemplary embodiment of an absorbent article.

FIG. 13 is a top down view of an exemplary embodiment of an absorbent article.

FIG. 14A is an exemplary embodiment of a cross-sectional view of the absorbent article of FIG. 13 taken along line 14A-14A.

FIG. 14B is an exemplary embodiment of a cross-sectional view of the absorbent article of FIG. 13 taken along line 14B-14B.

FIG. 15A is an exemplary embodiment of a cross-sectional view of the absorbent article of FIG. 13 taken along line 15A-15A.

FIG. 15B is an exemplary embodiment of a cross-sectional view of the absorbent article of FIG. 13 taken along line 15B-15B.

FIG. 16 is a top down view of an exemplary embodiment of an absorbent article.

FIG. 17 is a top down view of an exemplary embodiment of an absorbent article.

FIG. 18A is an exemplary embodiment of a cross-sectional view of the absorbent article of FIG. 17 taken along line 18A-18A.

FIG. 18B is an exemplary embodiment of a cross-sectional view of the absorbent article of FIG. 17 taken along line 18B-18B.

FIG. 19A is an exemplary embodiment of a cross-sectional view of the absorbent article of FIG. 17 taken along line 19A-19A.

FIG. 19B is an exemplary embodiment of a cross-sectional view of the absorbent article of FIG. 17 taken along line 19B-19B.

FIG. 20 is a top down view of an exemplary embodiment of an absorbent article.

Repeat use of reference characters in the present specification and drawings is intended to represent the same or analogous features or elements of the disclosure.

### DETAILED DESCRIPTION OF THE DISCLOSURE

The present disclosure is directed towards an absorbent article which can have a liquid permeable topsheet layer, a liquid impermeable backsheet layer, and an absorbent core positioned between the liquid permeable topsheet layer and the liquid impermeable backsheet layer. The absorbent core can have a topsheet layer facing surface and a backsheet layer facing surface, a primary absorbent region having a first height measured from the topsheet layer facing surface to the backsheet layer facing surface and a first density, a secondary absorbent region having a second height measured from the topsheet layer facing surface to the backsheet layer facing surface and a second density, and a tertiary absorbent region having a third height measured from the topsheet layer facing surface to the backsheet layer facing surface and a third density. The first height of the absorbent core is greater than the second height of the absorbent core and the second height of the absorbent core is greater than the third height of the absorbent core. The first density of the absorbent core is the same as the second density of the absorbent core. The third density of the absorbent core is greater than each of the first density and second density of the absorbent core.

### Definitions

As used herein, the term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, training pants, youth pants, swim pants, feminine hygiene products including, but not limited to, menstrual pads, sanitary napkins, feminine pads, pantiliners, and panty shields, and incontinence products, and the like without departing from the scope of the present disclosure.

As used herein, the term “airlaid” refers herein to a web manufactured by an airlaying process. In the airlaying process, bundles of small fibers having typical lengths ranging from about 3 to about 52 mm are separated and entrained in an air supply and then deposited onto a forming screen, usually with the assistance of a vacuum supply. The randomly deposited fibers are then bonded to one another using, for example, hot air to activate a binder component or a latex adhesive. Airlaying is taught in, for example, U.S. Pat. No. 4,640,810 to Laursen, et al., which is incorporated herein in its entirety by reference thereto for all purposes.

As used herein, the term “bonded” refers to the joining, adhering, connecting, attaching, or the like, of two elements. Two elements will be considered bonded together when they are joined, adhered, connected, attached, or the like, directly to one another or indirectly to one another, such as when bonded to an intermediate element. The bonding can occur via, for example, adhesive, pressure bonding, thermal bonding, ultrasonic bonding, stitching, suturing, and/or welding.

As used herein, the term “bonded carded web” refers herein to webs that are made from staple fibers which are sent through a combing or carding unit which separates or breaks apart and aligns the staple fibers in the machine direction to form a generally machine direction oriented fibrous nonwoven web. This material may be bonded together by methods that can include point bonding, through air bonding, ultrasonic bonding, adhesive bonding, etc.

As used herein, the term “conjugate fibers” refers herein to fibers which have been formed from at least two polymer sources extruded from separate extruders and spun together to form on fiber. Conjugate fibers are also sometimes referred to as bicomponent or multicomponent fibers. The polymers are arranged in substantially constantly positioned distinct zones across the cross-sections of the conjugate fibers and extend continuously along the length of the conjugate fibers. The configuration of such a conjugate fiber may be, for example, a sheath/core arrangement where one polymer is surrounded by another, or may be a side-by-side arrangement, a pie arrangement, or an “islands-in-the-sea” arrangement. Conjugate fibers are taught by U.S. Pat. No. 5,108,820 to Kaneko, et al., U.S. Pat. No. 4,795,668 to Krueger, et al., U.S. Pat. No. 5,540,992 to Marcher, et al., U.S. Pat. No. 5,336,552 to Strack, et al., U.S. Pat. No. 5,425,987 to Shawver, and U.S. Pat. No. 5,382,400 to Pike, et al., each being incorporated herein in their entirety by reference thereto for all purposes. For two component fibers, the polymers may be present in ratios of 75/25, 50/50, 25/75 or any other desired ratio. Additionally, polymer additives such as processing aids may be included in each zone.

As used herein, the term “machine direction” (MD) refers to the length of a fabric in the direction in which it is produced, as opposed to a “cross-machine direction” (CD) which refers to the width of a fabric in a direction generally perpendicular to the machine direction.

As used herein, the term “meltblown web” refers herein to a nonwoven web that is formed by a process in which a molten thermoplastic material is extruded through a plurality of fine, usually circular, die capillaries as molten fibers into converging high velocity gas (e.g., air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Buten, et al., which is incorporated herein in its entirety by reference thereto for all purposes. Generally speaking, meltblown fibers may be microfibers that are substantially continuous or discontinuous, generally smaller than 10 microns in diameter, and generally tacky when deposited onto a collecting surface.

As used herein, the term “nonwoven fabric” or “nonwoven web” refers herein to a web having a structure of individual fibers or threads which are interlaid, but not in an identifiable manner as in a knitted fabric. Nonwoven fabrics or webs have been formed from many processes such as, for example, meltblowing processes, spunbonding processes, through-air bonded carded web (also known as BCW and TABCW) processes, etc. The basis weight of nonwoven webs may generally vary, such as, from about 5, 10, or 20 gsm to about 120, 125, or 150 gsm.

As used herein, the term “spunbond web” refers herein to a web containing small diameter substantially continuous fibers. The fibers are formed by extruding a molten thermoplastic material from a plurality of fine, usually circular, capillaries of a spinneret with the diameter of the extruded fibers then being rapidly reduced as by, for example, eductive drawing and/or other well-known spunbonding mechanisms. The production of spunbond webs is described and illustrated, for example, in U.S. Pat. No. 4,340,563 to Appel, et al., U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., and U.S. Pat. No. 5,382,400 to Pike, et al., which are each incorporated herein in their entirety by reference thereto for all purposes. Spunbond fibers are generally not tacky when they are deposited onto a collecting surface. Spunbond fibers may sometimes have diameters less than about 40 microns, and often between about 5 to about 20 microns.

As used herein, the terms “superabsorbent polymer,” “superabsorbent,” or “SAP” shall be used interchangeably and shall refer to polymers that can absorb and retain extremely large amounts of a liquid relative to their own mass. Water absorbing polymers, which are classified as hydrogels, which can be cross-linked, absorb aqueous solutions through hydrogen bonding and other polar forces with water molecules. A SAP's ability to absorb water is based in par on iconicity (a factor of the ionic concentration of the aqueous solution), and the SAP functional polar groups that have an affinity for water. SAP are typically made from the polymerization of acrylic acid blended with sodium hydroxide I the presence of an initiator to form a poly-acrylic acid sodium salt (sometimes referred to as sodium polyacrylate). Other materials are also used to make a superabsorbent polymer, such as polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile. SAP may be present in absorbent articles in particle or fibrous form or as a coating or another material or fiber.

Absorbent Article:

The present disclosure is directed towards an absorbent article which can have a liquid permeable topsheet layer, a liquid impermeable backsheet layer, and an absorbent core positioned between the liquid permeable topsheet layer and the liquid impermeable backsheet layer. The absorbent core can have a topsheet layer facing surface and a backsheet layer facing surface, a primary absorbent region having a first height measured from the topsheet layer facing surface to the backsheet layer facing surface and a first density, a secondary absorbent region having a second height measured from the topsheet layer facing surface to the backsheet layer facing surface and a second density, and a tertiary absorbent region having a third height measured from the topsheet layer facing surface to the backsheet layer facing surface and a third density. The first height of the absorbent core is greater than the second height of the absorbent core and the second height of the absorbent core is greater than the third height of the absorbent core. The first density of the absorbent core is the same as the second density of the absorbent core. The third density of the absorbent core is greater than each of the first density and second density of the absorbent core.

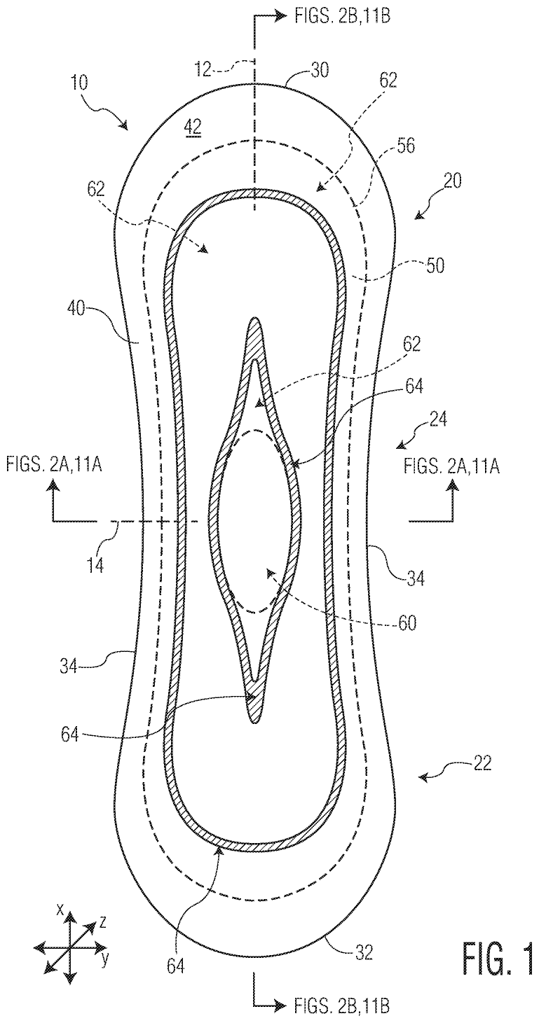

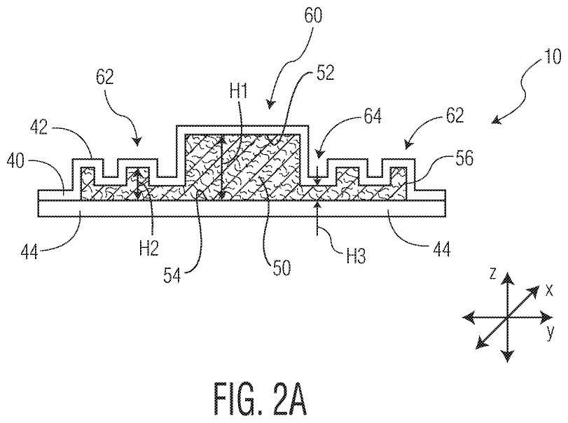

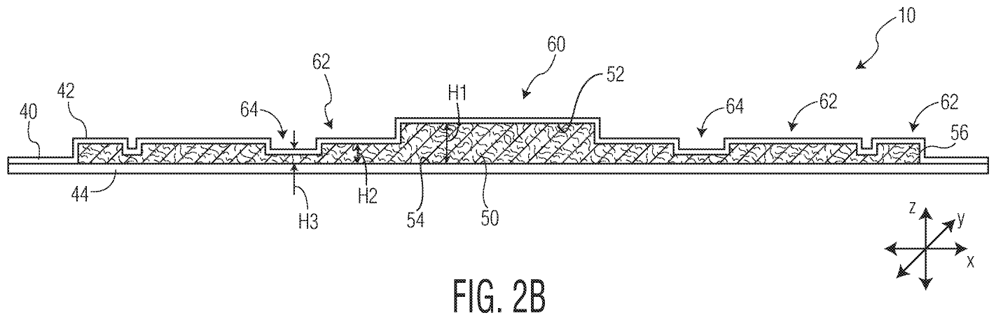

Referring to FIGS. 1, 2A and 2B, an absorbent article 10 of the present disclosure is exemplified in the form of a feminine hygiene product such as a menstrual pad or sanitary napkin. FIG. 1 provides a top down view of the exemplary embodiment of the absorbent article 10, FIG. 2A provides an exemplary embodiment of a cross-sectional view of the absorbent article 10 of FIG. 1 taken along line 2A-2A, and FIG. 2B provides an exemplary embodiment of a cross-sectional view of the absorbent article 10 of FIG. 1 taken along line 2B-2B. The absorbent article 10 can have a longitudinal direction (X), a transverse direction (Y), and a depth direction (Z). The absorbent article 10 can have a longitudinal axis 12 and a transverse axis 14. The absorbent article 10 can have an anterior region 20, a posterior region 22, and a crotch region 24 positioned between the anterior region 20 and the posterior region 22. When the absorbent article 10 is in use, the crotch region 24 can be positioned at the primary location of exudate discharge from the wearer of the absorbent article 10.

The absorbent article 10 can have a first transverse direction end edge 30, a second transverse direction end edge 32 opposed to the first transverse direction end edge 30, and a pair of opposing longitudinal direction side edges 34 extending between and connecting the first and second transverse direction end edges, 30 and 32. The absorbent article 10 can have a wearer facing, liquid permeable topsheet layer 40 and a garment facing, liquid impermeable backsheet layer 44. An absorbent core 50 can be positioned between the topsheet layer 40 and the backsheet layer 44. The topsheet layer 40 and the backsheet layer 44 can both extend beyond the outermost perimeter edge 56 of the absorbent core 50 and can be peripherally bonded together using known bonding techniques to form a sealed peripheral region. For example, the topsheet layer 40 and the backsheet layer 44 can be bonded together by adhesive bonding, ultrasonic bonding, or any other suitable bonding technique known in the art.

Each of these components of the absorbent article 10, as well as additional components, will be described in more detail herein.

Topsheet Layer:

The topsheet layer 40 defines a body facing surface 42 of the absorbent article 10 that may directly contact the body of the wearer and is liquid permeable to receive body exudates. The topsheet layer 40 is desirably provided for comfort and functions to direct body exudates away from the body of the wearer, through its own structure, and towards the absorbent core 50. The topsheet layer 40 desirably retains little to no liquid in its structure, so that it provides a relatively comfortable and non-irritating surface next to the skin of the wearer of the absorbent article 10.

The topsheet layer 40 can be a single layer of material, or alternatively, can be multiple layers that have been laminated together. The topsheet layer 40 can be constructed of any material such as one or more woven sheets, one or more fibrous nonwoven sheets, one or more film sheets, such as blown or extruded films, which may themselves be of single or multiple layers, one or more foam sheets, such as reticulated, open cell or closed cell foams, a coated nonwoven sheet, or a combination of any of these materials. Such combination can be adhesively, thermally, or ultrasonically laminated into a unified planar sheet structure to form a topsheet layer 40.

In various embodiments the topsheet layer 40 can be constructed from various nonwoven webs such as meltblown webs, spunbond webs, hydroentangled spunlace webs, or through air bonded carded webs. Examples of suitable topsheet layer 40 materials can include, but are not limited to, natural fiber webs (such as cotton), rayon, hydroentangled webs, bonded carded webs of polyester, polypropylene, polyethylene, nylon, or other heat-bondable fibers (such as bicomponent fibers), polyolefins, copolymers of polypropylene and polyethylene, linear low-density polyethylene, and aliphatic esters such as polylactic acid. Finely perforated films and net materials can also be used, as can laminates of/or combinations of these materials. An example of a suitable topsheet layer 40 can be a bonded carded web made of polypropylene and polyethylene such as that obtainable from Sandler Corp., Germany. U.S. Pat. No. 4,801,494 to Datta, et al., and U.S. Pat. No. 4,908,026 to Sukiennik, et al., and WO 2009/062998 to Texol teach various other topsheet materials that may be used as the topsheet layer 40, each of which is hereby incorporated by reference thereto in its entirety. Additional topsheet layer 40 materials can include, but are not limited to, those described in U.S. Pat. No. 4,397,644 to Matthews, et al., U.S. Pat. No. 4,629,643 to Curro, et al., U.S. Pat. No. 5,188,625 to Van Iten, et al., U.S. Pat. No. 5,382,400 to Pike, et al., U.S. Pat. No. 5,533,991 to Kirby, et al., U.S. Pat. No. 6,410,823 to Daley, et al., and U.S. Publication No. 2012/0289917 to Abuto, et al., each of which is hereby incorporated by reference thereto in its entirety.

In various embodiments, the topsheet layer 40 may contain a plurality of apertures formed therethrough to permit body exudates to pass more readily into the absorbent core 50. The apertures may be randomly or uniformly arranged throughout the topsheet layer 40. The size, shape, diameter, and number of apertures may be varied to suit an absorbent article's 10 particular needs.

In various embodiments, the tospheet layer 40 can have a basis weight ranging from about 5, 10, 15, 20, or 25 gsm to about 50, 100, 120, 125, or 150 gsm. For example, in an embodiment, a topsheet layer 40 can be constructed from a through air bonded carded web having a basis weight ranging from about 15 gsm to about 100 gsm. In another example, a topsheet layer 40 can be constructed from a through air bonded carded web having a basis weight from about 20 gsm to about 50 gsm, such as a through air bonded carded web that is readily available from nonwoven material manufacturers, such as Xiamen Yanjan Industry, Beijing, DaYuan Nonwoven Fabrics, and others.

In various embodiments, the topsheet layer 40 can be at least partially hydrophilic. In various embodiments, a portion of the topsheet layer 40 can be hydrophilic and a portion of the topsheet layer 40 can be hydrophobic. In various embodiments, the portions of the topsheet layer 40 which can be hydrophobic can be either an inherently hydrophobic material or can be a material treated with a hydrophobic coating.

In various embodiments, the topsheet layer 40 can be a multicomponent topsheet layer 40 such as by having two or more different nonwoven or film materials, with the different materials placed in separate locations in the transverse direction (Y) of the absorbent article 10. For example, the topsheet layer 40 can be a two layer or multicomponent material having a central portion positioned along and straddling a longitudinal axis 12 of an absorbent article 10, with lateral side portions flanking and bonded to each side edge of the central portion. The central portion can be constructed from a first material and the side portions can be constructed from a material which can be the same as or different from the material of the central portion. In such embodiments, the central portion may be at least partially hydrophilic and the side portions may be inherently hydrophobic or may be treated with a hydrophobic coating. Examples of constructions of multi-component topsheet layers 40 are generally described in U.S. Pat. No. 5,961,505 to Coe, U.S. Pat. No. 5,415,640 to Kirby, and U.S. Pat. No. 6,117,523 to Sugahara, each of which is incorporated herein by reference thereto in its entirety.

In various embodiments, a central portion of a topsheet layer 40 can be positioned symmetrically about the absorbent article 10 longitudinal axis 12. Such central longitudinally directed central portion can be a through air bonded carded web (“TABCW”) having a basis weight between about 15 and about 100 gsm. Previously described nonwoven, woven, and aperture film topsheet layer materials may also be used as the central portion of a topsheet layer 40. In various embodiments, the central portion can be constructed from a TABCW material having a basis weight from about 20 gsm to about 50 gsm such as is available from Xiamen Yanjan Industry, Beijing, DaYuan Nonwoven Fabrics, and others. Alternatively, aperture films, such as those available from such film suppliers as Texol, Italy and Tredegar, U.S.A. may be utilized. Different nonwoven, woven, or film sheet materials may be utilized as the side portions of the topsheet layer 40. The selection of such topsheet layer 40 materials can vary based upon the overall desired attributes of the topsheet layer 40. For example, it may be desired to have a hydrophilic material in the central portion and hydrophobic-barrier type materials in the side portions to prevent leakage and increase a sense of dryness in the area of the side portions. Such side portions can be adhesively, thermally, ultrasonically, or otherwise bonded to the central portion along or adjacent the longitudinally directed side edges of the central portion. Traditional absorbent article construction adhesive may be used to bond the side portions to the central portion. Either of the central portion and/or the side portions may be treated with surfactants and/or skin-health benefit agents, as are well known in the art.

Such longitudinally directed side portions can be of a single or multi-layered construction. In various embodiments, the side portions can be adhesively or otherwise bonded laminates. In various embodiments, the side portions can be constructed of an upper fibrous nonwoven layer, such as a spunbond material, laminated to a bottom layer of a hydrophobic barrier film material. Such a spunbond layer may be formed from a polyolefin, such as a polypropylene and can include a wetting agent if desired. In various embodiments, a spunbond layer can have a basis weight from about 10 or 12 gsm to about 30 or 70 gsm and can be treated with hydrophilic wetting agents. In various embodiments, a film layer may have apertures to allow fluid to permeate to lower layers, and may be either of a single layer or multi-layer construction. In various embodiments, such film can be a polyolefin, such as polyethylene having a basis weight from about 10 to about 40 gsm. Construction adhesive can be utilized to laminate the spunbond layer to the film layer at an add-on level of between about 0.1 gsm and 15 gsm. When a film barrier layer is used in the overall topsheet layer 40 design, it may include opacifying agents, such as film pigments, that can help the film in masking stains along the absorbent article 10 side edges, thereby serving as a masking element. In such a fashion, the film layer can serve to limit visualization of a fluid insult stain along the absorbent article 10 side edges when viewed from above the topsheet layer 40. The film layer may also serve as a barrier layer to prevent rewet of the topsheet layer 40 as well as to prevent the flow of fluid off the side edges of the absorbent article 10. In various embodiments, the side portions can be laminates such as a spunbond-meltblown-meltblown-spunbond layer (“SMMS”) laminate, spunbond-film laminate, or alternatively, other nonwoven laminate combinations.

Absorbent Core:

The absorbent article 10 can have an absorbent core 50 positioned between the topsheet layer 40 and the backsheet layer 44. The absorbent core 50 is designed to absorb body exudates, including menstrual fluid, blood, urine, and other bodily fluids such as sweat and vaginal discharge. The absorbent core 50 can generally be any single layer structure which can demonstrate some level of compressibility, conformability, be non-irritating to a wearer's skin, and capable of absorbing and retaining liquids and other body exudates.

In various embodiments, the absorbent core 50 can be formed from a variety of different materials. For example, the absorbent core 50 can include a layer of absorbent material of cellulosic fibers (e.g., wood pulp fibers), other natural fibers, synthetic fibers, woven or nonwoven sheets, scrim netting, or other stabilizing structures, superabsorbent material, binder materials, surfactants, selected hydrophobic and hydrophilic materials, pigments, lotions, odor control agents or the like, as well as combinations thereof. In various embodiments, the absorbent material can include a matrix of cellulosic fluff. In various embodiment, the absorbent material can include a matrix of cellulosic fluff and can also include superabsorbent material. The cellulosic fluff can comprise a blend of wood pulp fluff. An example of a wood pulp fluff can be identified with the trade designation NB 416, available from Weyerhaeuser Corp., and is a bleached, highly absorbent wood pulp containing primarily soft wood fibers.

In various embodiments, if desired, the absorbent core 50 can include an optional amount of superabsorbent material. Examples of suitable superabsorbent material can include poly(acrylic acid), poly(methacrylic acid), poly(acrylamide), poly(vinyl ether), maleic anhydride copolymers with vinyl ethers and α-olefins, poly(vinyl pyrrolidone), poly(vinylmorpholinone), poly(vinyl alcohol), and salts and copolymers thereof. Other superabsorbent materials can include unmodified natural polymers and modified natural polymers, such as hydrolyzed acrylonitrile-grafted starch, acrylic acid grafted starch, methyl cellulose, chitosan, carboxymethyl cellulose, hydroxypropyl cellulose, and natural gums, such as alginates, xanthan gum, locust bean gum, and so forth. Mixtures of natural and wholly or partially synthetic superabsorbent polymers can also be useful. The superabsorbent material can be present in the absorbent core 50 in any amount as desired.

The absorbent core 50 can be provided in any shape, as defined by the perimeter edge 56, as deemed suitable for the absorbent article 10 such as, but not limited to, oblong, oval, rectangular, tear-dropped, dog-bone, hourglass, racetrack, triangular, and elliptical as well as any other geometric shape as deemed suitable for the absorbent article 10. In various embodiments, the shape of the absorbent core 50 can have a shape which provides symmetry about at least one axis, longitudinal 12 and/or transverse 14, of the absorbent article 10. In various embodiments, the shape of the absorbent core 50 can have a shape which provides symmetry about each axis, longitudinal 12 and transverse 12, of the absorbent article 10. In various embodiments, the shape of the absorbent core 50 can be one in which there is no symmetry of the absorbent core 50 about either of the axes, longitudinal 12 or transverse 14 of the absorbent article 10.

The absorbent core 50 can have a topsheet layer facing surface 52 and a backsheet layer facing surface 54. As measured from the topsheet layer facing surface 52 of the absorbent core 50 to the backsheet layer facing surface 54 of the absorbent core 50, the absorbent core 50 can have a variable height in the depth direction (Z). The absorbent core 50 can have a primary absorbent region 60 which has a first height H1 in the depth direction (Z) and a secondary absorbent region 62 which has a second height H2 in the depth direction (Z). The first height H1 of the primary absorbent region 60 is greater than the second height H2 of the secondary absorbent region 62. In various embodiments, the first height H1 can be at least about 6, 7, 8, 9, or 10 mm. In various embodiments, the first height H1 of the primary absorbent region 60 can be from about 6 or 7 mm to about 8, 9, or 10 mm. In various embodiments, the second height H2 of the secondary absorbent region 62 can be from about 25 or 50% to about 60 or 75% of the first height H1 of the primary absorbent region 60. In various embodiments, the second height H2 of the secondary absorbent region 62 can be from about 2 or 3 mm to about 4 or 5 mm.

At least a portion of the primary absorbent region 60 of the absorbent core 50 can be located within the crotch region 24 of the absorbent article 10. In various embodiments, the primary absorbent region 60 can have a first portion located within the crotch region 24 of the absorbent article 10 and can have a second portion located within either the anterior region 20 or the posterior region 22 of the absorbent article 10. In various embodiments, the primary absorbent region 60 can have a first portion located within the crotch region 24, a second portion located within the anterior region 20, and a third portion located within the posterior region 22 of the absorbent article 10. In various embodiments, the primary absorbent region 60 can traverse the transverse axis 14 of the absorbent article 10 whereby portions of the primary absorbent region 60 are located on opposing sides of the transverse axis 14 of the absorbent article 10. In various embodiments, the entire primary absorbent region 60 is located on only one side of the transverse axis 14 of the absorbent article 10 whereby the primary absorbent region 60 does not traverse the transverse axis 14 of the absorbent article 10. In various embodiments, the primary absorbent region 60 can traverse the longitudinal axis 12 of the absorbent article 10 whereby portions of the primary absorbent region 60 are located on opposing sides of the longitudinal axis 12 of the absorbent article 10. In various embodiments, the entire primary absorbent region 60 is located on only one side of the longitudinal axis 12 of the absorbent article 10 whereby the primary absorbent region 60 does not traverse the longitudinal axis 12 of the absorbent article 10. In various embodiments, the primary absorbent region 60 traverses each of the longitudinal axis 12 and the transverse axis 14 of the absorbent article 10. In various embodiments, the primary absorbent region 60 does not traverse either of the longitudinal axis 12 or the transverse axis 14 of the absorbent article 10.

In various embodiments, the primary absorbent region 60 can be adjacent to the secondary absorbent region 62. In various embodiments, the primary absorbent region 60 can be adjacent to the secondary absorbent region 62 in the longitudinal direction (X) of the absorbent core 50. In various embodiments, the primary absorbent region 60 can be adjacent to the secondary absorbent region 62 in the transverse direction (Y) of the absorbent core 50. In various embodiments, the primary absorbent region 60 can be adjacent to the secondary absorbent region 62 in each of the longitudinal direction (X) and the transverse direction (Y) of the absorbent core 50. In various embodiments, the primary absorbent region 60 is not adjacent to the secondary absorbent region 62.

In various embodiments in which the primary absorbent region 60 is adjacent to the secondary absorbent region 62, the transition from the primary absorbent region 60 to the secondary absorbent region 62 can be an abrupt transition. In various embodiments in which the primary absorbent region 60 is adjacent to the secondary absorbent region 62, the transition from the primary absorbent region 60 to the secondary absorbent region 62 can be a gradual transition.

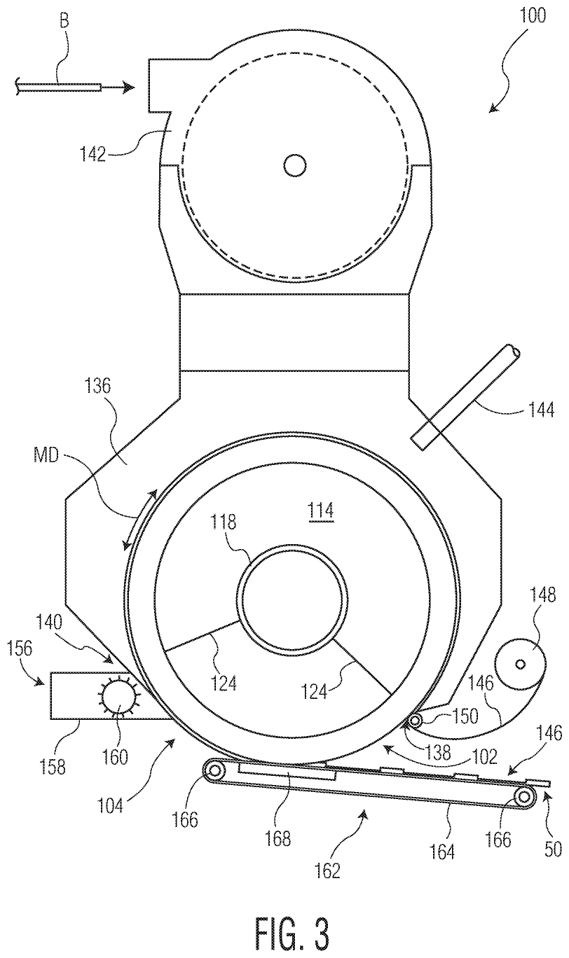

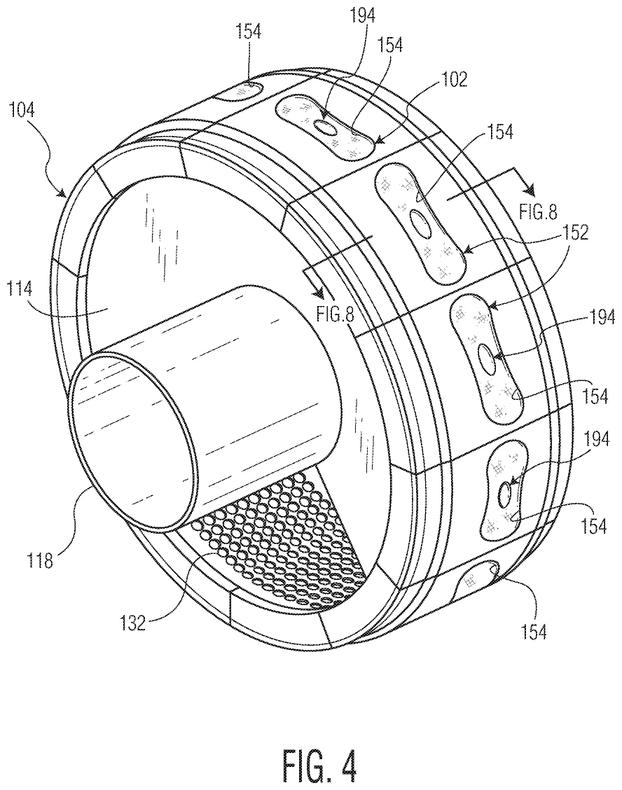

In various embodiments, to form an absorbent core 50 having a primary absorbent region 60 having a first height H1 and a secondary absorbent region 62 having a second height H2 which is less than the first height H1 of the primary absorbent region 60, the absorbent core 50 can be formed by an airlaying technique. For example, referring to FIGS. 3-5, for purposes of the present description the apparatus has a machine-direction (MD) which extends generally in the direction of motion of the machine, a lateral cross-machine direction (CD) which extends transversely to the machine-direction (MD), and a z-direction (Z). The machine-direction (MD) is the direction along which a particular component or material is transported lengthwise along and through a particular, local position of the apparatus. The cross-machine direction (CD) lies generally within the plane of the material being transported through the process and is transverse to the local machine-direction (MD). The z-direction (Z) is aligned substantially perpendicular to both the machine-direction (MD) and the cross-machine direction (CD), and extends generally along a depth-wise, thickness dimension of the material.

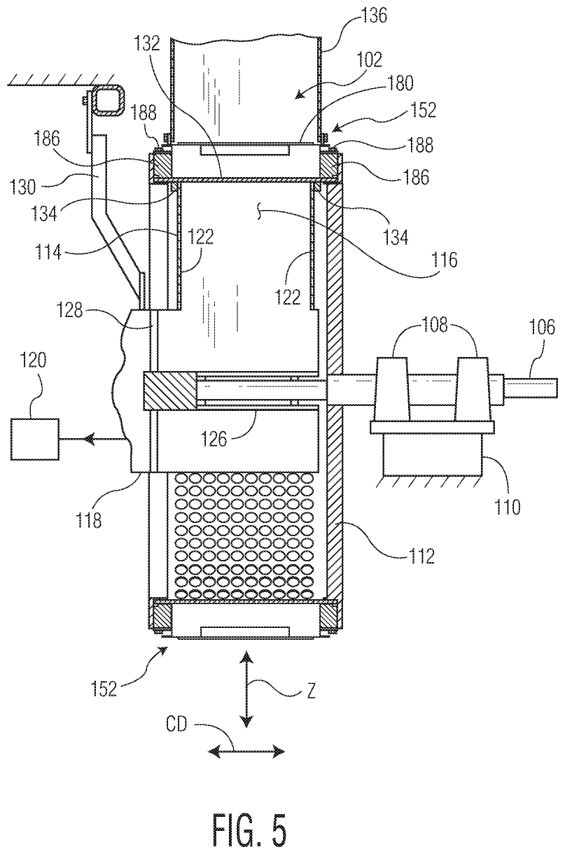

An apparatus 100 for forming the absorbent core 50 can include a movable, foraminous forming surface 102 extending around the circumference of a drum 104. The drum 104 is mounted on a shaft 106 connected by bearings 108 to a support 110. As shown in FIG. 5, the drum 104 includes a circular wall 112 connected to the shaft 106 for conjoint rotation therewith. The shaft 106 is driven in rotation by a suitable motor or line shaft in a counterclockwise direction as illustrated in FIG. 3. The circular wall 112 cantilevers the forming surface 102 and the opposite side of the drum 104 is open. A vacuum duct 114 located radially inwardly of the forming surface 102 extends over an arc of the drum interior. The vacuum duct 114 has an arcuate, elongate entrance opening 116 under the forming surface 102 for fluid communication with a vacuum supply conduit 118 connected to a vacuum source 120 (represented diagrammatically in FIG. 5). The vacuum source 120 may be, for example, an exhaust fan. The vacuum duct 114 is connected to the vacuum supply conduit 118 along an outer peripheral surface of the vacuum supply conduit 118 and extends circumferentially of the vacuum supply conduit 118. The vacuum duct 114 projects radially outwardly from the vacuum supply conduit 118 toward the forming surface 102 and includes axially spaced side walls 122 and angularly spaced end walls 124. The shaft 106 extends through the circular wall 112 and into the vacuum supply conduit 118 where it is received in a bearing 126 connected to a brace 128 within the vacuum supply conduit 118. The bearing 126 is sealed with the vacuum supply conduit 118 so that air is not drawn in around the shaft 106 where it enters the vacuum supply conduit 118. The brace 128 and entire vacuum supply conduit 118 are supported by an overhead mount 130.

A drum rim 132 is mounted on the circular wall 112 of the drum 104 and has a multiplicity of holes over its surface area to provide a substantially free movement of air through the thickness of the drum rim 132. The drum rim 132 is generally tubular in shape and extends around the axis of rotation of the shaft 106 near the periphery of the circular wall 112. The drum rim 132 is cantilevered away from the circular wall 112 and has a radially inward-facing surface positioned closely adjacent to the entrance opening 116 of the vacuum duct 114. To provide an air resistant seal between the drum rim 132 and the entrance opening 116 of the vacuum duct 114, rim seals 134 are mounted on the inward-facing surface of the drum rim 132 for sliding sealing engagement with the side walls 122 of the vacuum duct 114. Seals are also mounted on the end walls 124 of the vacuum duct 114 for sliding sealing engagement with the inward-facing surface of the drum rim 132. The seals may be formed of a suitable material such as felt to permit the sliding sealing engagements.

The apparatus 100 further includes a forming chamber 136 through which the forming surface 102 is movable. The forming chamber 136 has an entrance 138 where the forming surface 102 enters the forming chamber 136 substantially free of absorbent material, and an exit 140 where the forming surface 102 leaves the forming chamber 136 substantially filled with absorbent material. A fiberizer 142 provides fibrous material into the forming chamber 136 and the vacuum source 120 creates a vacuum pressure in the vacuum duct 114 relative to the interior of the forming chamber 136. As the forming surface 102 enters and then traverses through the forming chamber 136 the component materials of the absorbent core 50 are operatively carried or transported by an entraining air stream that is drawn through the forming surface 102. The pressure differential across the forming surface 102 causes the fluent fibers in the forming chamber 136 to be drawn to the forming surface 102.

As described herein, in various embodiments, the absorbent material of the absorbent core 50 can be derived from a batt B of cellulosic fibers (e.g., wood pulp fibers), other natural fibers, and/or synthetic fibers, which have been disintegrated, in a manner known in the art, to provide an operative quantity of individual, loose fibers. The fiberizer 142 receives a selected absorbent core-forming material, converts the absorbent core-forming material into individual fibers, and delivers the fibers into the forming chamber 136. In the illustrated configuration, the fiberizer 142 can be a rotary hammer mill or a rotatable picker roll. However, it is to be understood that fibers may be provided in other ways by other devices within the scope of the present disclosure.

Other component materials for producing the absorbent core 50 may also be delivered into the forming chamber 136. For example, particles or fibers of superabsorbent material (such as described herein) may be introduced into the forming chamber 136 by employing conventional mechanisms, such as pipes, channels, spreaders, nozzles, and the like, as well as combinations thereof. In the illustrated embodiment, the superabsorbent material is delivered into the forming chamber 136 by employing a schematically represented delivery conduit and nozzle system 144. The fibers, superabsorbent material, and other desired absorbent core material may be entrained in any suitable gaseous medium. Accordingly, any references herein to air as being the entraining medium should be understood to be a general reference which encompasses any other operative entraining gas.

The stream of fluent absorbent material pass through the forming chamber 136 for deposition on the forming surface 102. The forming chamber 136 can serve to direct and concentrate the air-entrained absorbent material and to provide a desired velocity profile in the air-entrained stream of absorbent material. Typically, the forming chamber 136 is supported by suitable structural members which together form a support frame for the forming chamber 136. The frame may be anchored and/or joined to other suitable structural components, as necessary or desirable. The construction and operation of such forming chambers 136 is well known and will not be described in further detail herein. Instead of applying the absorbent material directly to the forming surface 102, it is known to place a porous substrate over the forming surface 102 on which the absorbent materials are deposited. In various embodiments, the porous substrate can be any material deemed suitable for forming the topsheet layer 40 as described herein. A web 146 of porous substrate is shown in phantom in FIG. 3 to extend from a roll 148 into the entrance 138 of the forming chamber 136. The roll 148 can be held and the web 146 fed out by suitable delivery device (not shown in its entirety) as is known in the art. A roller 150 of the delivery device is shown for guiding the web 146 into the entrance 138. The web 146 overlies the forming surface 102 so that absorbent materials are deposited on the web 146 rather than directly on the forming surface 102. The vacuum causes the web 146 to conform to the shape of the forming surface 102. The use of such a web 146 desirably reduces the amount of absorbent material which passes completely through the forming surface 102 because individual pores of the web 146 are smaller than the openings in the forming surface 102. However, for simplicity, the illustrated embodiment will be described hereinafter without reference to the web 146.

The forming surface 102 is illustrated as being part of the drum 104, but it is to be understood that other techniques for providing the forming surface 102 may also be employed without departing from the scope of the present disclosure. For example, the forming surface 102 may be provided by an endless forming belt.

The forming surface 102 is defined in the illustrated embodiment by a series of form members 152 which are arranged end-to-end around the periphery of the drum 104 and independently attached to the drum 104. The form members 152 each define a substantially identical pattern 154 in which absorbent material is deposited. The patterns 154 correspond to a desired shape of individual absorbent cores 50 which repeats over the circumference of the drum. However, partially repeating or non-repeating pattern shapes may be used with the present disclosure. Under the influence of the vacuum source 120, a conveying air stream is drawn through the forming surface 102 into the vacuum duct 114 on the interior of the drum 104 and is subsequently passed out of the drum 104 through the vacuum supply conduit 118. As the absorbent material impinge the forming surface 102, the air component is passed through the forming surface 102 and the absorbent material are retained by the forming surface 102 to form an absorbent core 50 thereon. Subsequently with rotation of the drum 104, the absorbent core 50 is removed from the forming surface 102.

The drum 104 carrying the air formed absorbent core 50 deposited on the forming surface 102 in the forming chamber 136 passes out of the forming chamber 136 through the exit 140 to a scarfing system 156 where excess thickness of the absorbent core 50 can be trimmed and removed to a predetermined extent. The scarfing system 156 includes a scarfing chamber 158 and a scarfing roll 160 which is positioned within the scarfing chamber 158. The scarfing roll 160 abrades excess absorbent material from the absorbent core 50 and the removed fibers are transported away from the scarfing chamber 158 with a suitable discharge conduit (not shown), as well known in the art. The removed absorbent material may, for example, be recycled back into the forming chamber 136 or the fiberizer 142, as desired. Additionally, the scarfing roll 160 can rearrange and redistribute the absorbent material along the longitudinal machine direction (MD) of the absorbent core 50 and/or along the lateral cross-machine direction (CD) of the absorbent core 50. The profile of the absorbent core 50 made by a scarfing roll 160 may be flat but may also be shaped or irregular as desired by selection and arrangement of teeth (not shown) on the scarfing roll 160.

The rotatable scarfing roll 160 is operatively connected and joined to a suitable shaft member and is driven by a suitable drive system (not shown). The drive system may include any conventional apparatus, such as provided by a dedicated motor, or a coupling, gear or other transmission mechanism operatively connected to the motor or other drive mechanism employed to rotate the drum 104. The scarfing system 156 can provide a conventional trimming mechanism for removing or redistributing any excess, z-directional thickness of the air formed absorbent core 50 that has been deposited on the forming surface 102. The scarfing operation can yield an absorbent core 50 having a selected contour on a major surface of the absorbent core 50 that has been contacted by the scarfing roll 160. The surface of the scarfing roll 160 can be adjusted to provide a desired contour along the scarfed surface of the absorbent core 50. In the illustrated embodiment, the scarfing roll 160 can, for example, be configured to provide a substantially flat surface along the scarfed surface of the absorbent core 50. The scarfing roll 160 can optionally be configured to provide a non-flat surface. The scarfing roll 160 is disposed in a spaced adjacent relationship to the forming surface 102, and the forming surface 102 is translated past the scarfing roll 160 by rotation of the drum 104.

In the illustrated embodiment, the scarfing roll 160 rotates in a direction which moves a contacting surface of the scarfing roll 160 in a counter-direction that is opposite the rotation of the drum 104 and the movement direction of the air formed absorbent core 50. Alternatively, the scarfing roll 160 may be rotated so that the scarfing roll 160 surface moves in the same direction as the forming surface 102 on the drum 104. In either situation, the rotational speed of the scarfing roll 160 should be suitably selected to provide an effective scarfing action against the contacted surface of the absorbent core 50. In like manner, any other suitable trimming mechanism may be employed in place of the scarfing roll 160 to provide a cutting or abrading action to the absorbent core 50 by a relative movement between the absorbent core 50 and the selected trimming mechanism.

After the scarfing operation, the portion of the forming surface 102 that is carrying the absorbent core 50 can be moved to a release zone of the apparatus 100. In the release zone vacuum causes the absorbent core 50 to transfer from the forming surface 102 onto a conveyor 162. The release can be assisted by the application of air pressure from the interior of the drum 104. The conveyor 162 receives the absorbent core 50 from the drum 104 and conveys the absorbent core 50 to a collection area or to a location for further processing (not shown). Suitable conveyors can, for example, include conveyor belts, vacuum drums, transport rollers, electromagnetic suspension conveyors, fluid suspension conveyors, or the like, as well as combinations thereof. In the illustrated embodiment, the conveyor 162 includes an endless conveyor belt 164 disposed about rollers 166. A vacuum suction box 168 is located below the conveyor belt 164 to remove the absorbent core 50 from the forming surface 102. The conveyor belt 164 is perforate and the vacuum suction box 168 defines a plenum beneath the portion of the conveyor belt 164 in close proximity to the forming surface 102 so that a vacuum is communicated to the absorbent core 50 on the drum 104. Removal of the absorbent core 50 can alternatively be accomplished by the weight of the absorbent core 50, by centrifugal force, by mechanical ejection, by positive air pressure, or by some combination or by another suitable method. The positive air pressure can be produced, for example, by a source of compressed air (not shown) such as a fan which generates a pressurized air flow that exerts a force directed outwardly through the forming surface 102. The removed absorbent core 50 includes an interconnected series of absorbent cores 50 (connected via the web 146 of porous substrate), and each absorbent core 50 has a selected surface contour which substantially matches the contour provided by the corresponding portions of the forming surface 102 upon which each individual absorbent core 50 was formed. It is also possible to contour the scarfed side of each absorbent core 50.

It will be understood that the description of the drum 104 shown in the Figures is exemplary, as other configurations (including those not having a drum for carrying the forming surface 102) may be employed to produce the absorbent core 50.

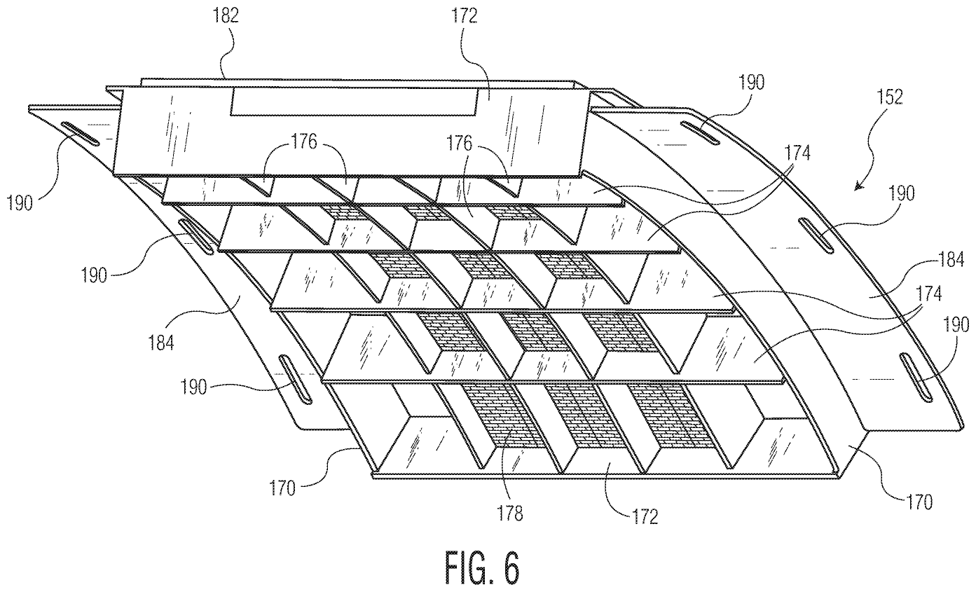

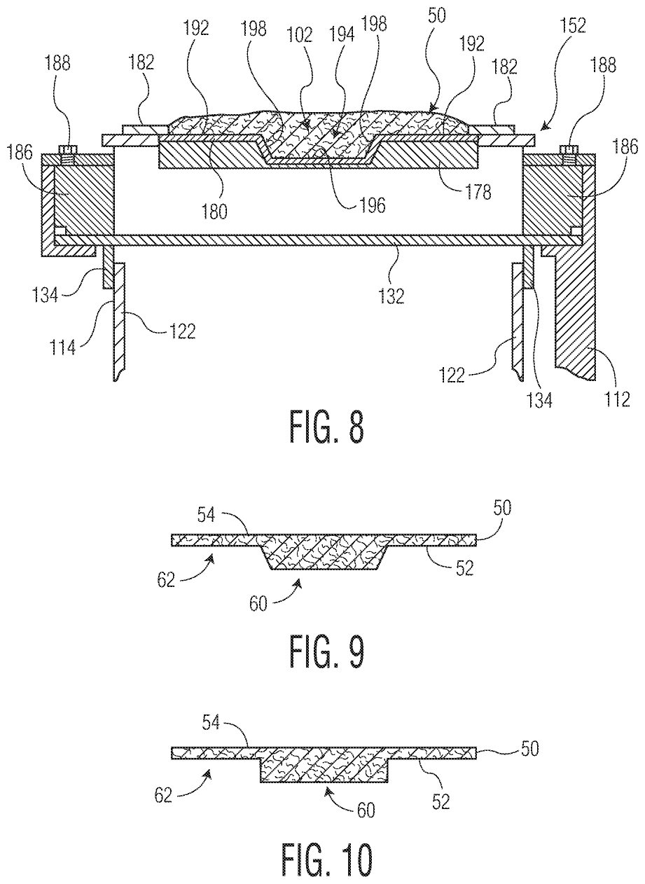

Referring to FIG. 6, a single form member 152 is shown as removed from the drum 104. As used herein, the term “form” can refer to a single form member 152 or to a collection of form members 152, such as the form members 152 which extend around the complete circumference of the drum 104. Moreover, it is envisioned that a single form member extending around the entire circumference of the drum 104 could be employed. The illustrated form member 152 comprises outer side walls 170 connected to end walls 172 to form a rectangular frame. The side walls 170 are curved along their length to match the arc of the drum 104 over which the individual form members 152 will extend. Transverse walls 174 extend between the side walls 170 and longitudinal walls 176 extend between the end walls 172 inside the frame. The frame supports the forming surface 102 which in the illustrated embodiment comprises a honeycombed support 178 and a thin, perforated plate 180 (see FIG. 8). The support 178 and perforated plate 180 have the same upper surface shape. The support 178 underlies and provides strength for the perforated plate 180 to hold it in a fixed configuration under the load applied by the vacuum. The support 178 permits air to pass freely through it by virtue of the relative larger openings of its honeycomb structure. The openings can have any desired cross-sectional shape, such as circular, oval, hexagonal, pentagonal, other polygonal shape or the like, as well as combinations thereof, and need not be in a honeycomb arrangement. Such support structures are well known in the art, and can be composed of various materials, such as plastic, metal, ceramics, and the like, as well as combinations thereof. The smaller holes in the perforated plate 180 also allow passage of air but are sized to capture the absorbent material and prevent its passing through the forming surface 102. The perforated plate 180 may be replaced by a screen, a wire mesh, a hard-wire cloth or the like, as well as combinations thereof. It is envisioned that if a sufficiently rigid, self-supporting material could be found for the perforated plate 180, the support 178 could be omitted.

A masking plate 182 is attached to the radially outwardly facing surface of the form member 152 to mask portions of the perforated plate 180 and support 178 to prevent air from passing through the masked portions and hence prevent deposition of absorbent material. The patterns 154 are defined by the shape of the masking plates 182. The form member 152 is mounted on the drum 104 by a pair of wings 184 attached to and extending laterally outward from respective side walls 170. When applied to the drum 104 as shown in FIG. 5, the wings 184 of the form member 152 overlie respective, axially spaced mounting rings 186 mounted on the drum rim 132 at its opposite lateral edges. The form member 152 is releasably secured to the mounting rings 186 by bolts 188 passing through elongate openings 190 in the wings 184 and threadably received in holes (not shown) forming in the mounting rings 186. The elongation of the openings 190 allows some variation in the circumferential position of the form member 152, facilitating placement of the form member 152 on the drum 104.

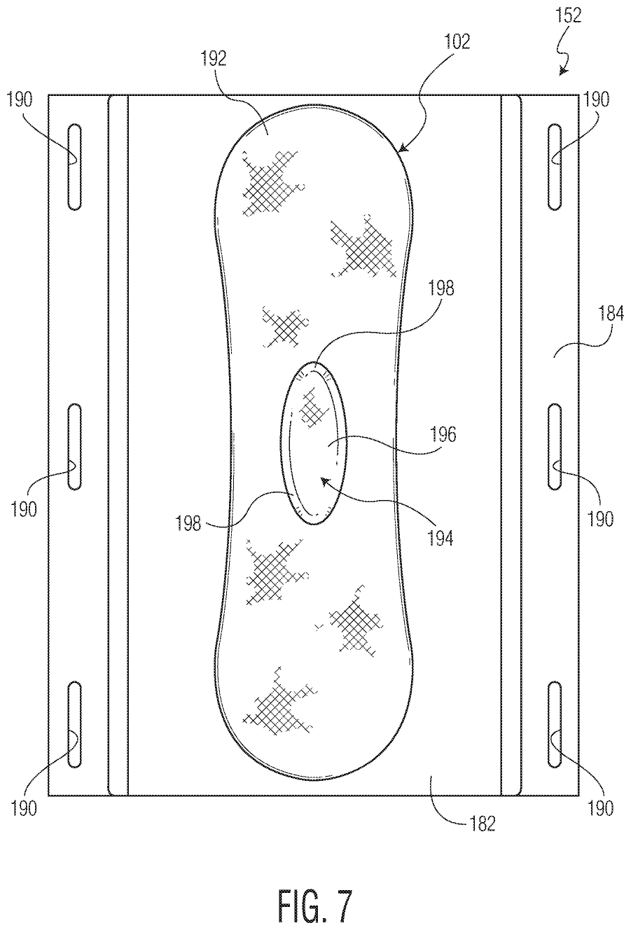

Referring now to FIGS. 7 and 8, a single form member 152 from the drum 104 is shown. The forming surface 102 has a length in the machine direction (MD) and a width in the cross-machine direction (CD) and is shaped to include a first section 192 at a first depth below the top surface of the masking plate 182. The first section 192 is relatively shallow and planar in configuration for forming a thinner layer of absorbent material. The first section 192 is curved between the ends of the form member 152 in correspondence with the curvature of the drum 104. Thus, rather than being truly planar, the first section 192 lies in a smooth surface and is substantially linear in cross section, as may be seen in FIG. 8.

A pocket 194 in the forming surface 102 includes a bottom surface 196 and a transition surface 198 connecting the first section 192 with the bottom surface 196 of the pocket 194. The absorbent material deposited into the pocket 194 will ultimately result in the primary absorbent region 60 of the absorbent core 50. The pocket 194 can have any shape as desired for a resultant primary absorbent region 60 such as, but not limited to, oblong, oval, rectangular, square, circle, tear-dropped, dog-bone, hourglass, racetrack, triangular, elliptical, as well as any other geometric shape as deemed suitable. The first section 192 can include portions lying on both sides of the bottom surface 196, and in the illustrated embodiment the first section 192 substantially surrounds the pocket 194. The bottom surface 196 of the pocket 194 has a generally flat configuration everywhere below the surface containing the first section 192 and is linear in cross section. The depth of the bottom surface 196 below the first section 192 is uniform over the area of the bottom surface 196. The depth of absorbent material deposited in the pocket 194 is greater than in the first section 192. In various embodiments it may be desired to have an absorbent core 50 which has more than one primary absorbent region 60. In such embodiments, the forming surface 102 can have more then one pocket 194 within which to deposit absorbent material that will ultimately form the primary absorbent regions 60 of the absorbent core 50.

In various embodiments, following the scarfing operation, the absorbent core 50 can appear like the absorbent core 50 illustrated in FIG. 9. In such embodiments, during the formation of the absorbent core 50, such as, for example, with the forming surface 102 illustrated in FIG. 8, the pocket 194 transition surface 198 connecting the bottom surface 196 of the pocket 194 to the first section 192 can be positioned at an angle. This can provide for an absorbent core 50 having a gradual transition between the primary absorbent region 60 and the secondary absorbent region 62. The topsheet layer facing surface 52 can be profiled due to the presence of the primary absorbent region 60 having a first height H1 which is greater than the secondary absorbent region 62 which has a second height H2. The backsheet layer facing surface 54 of the absorbent core 50 can be flat to not have any dip or ridge formed in the scarfed surface. Utilizing the airlaying method described herein to form the single layer absorbent core 50 having a primary absorbent region 60 and a secondary absorbent region 62 can result in each region, primary absorbent region 60 and secondary absorbent region 62, having the same density even though the heights between the two regions, primary absorbent region 60 and secondary absorbent region 62, are different.

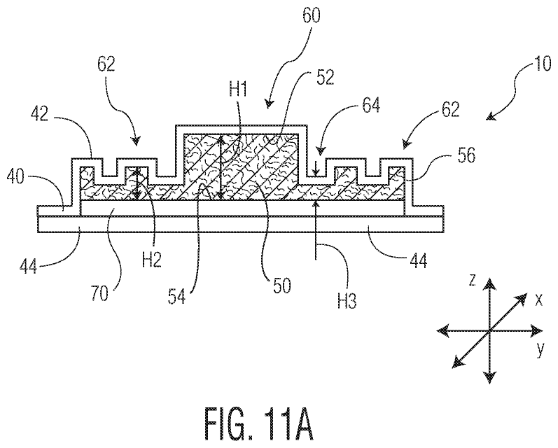

In various embodiments, following the scarfing operation, the absorbent core 50 can appear like the absorbent core 50 illustrated in FIG. 10. In such embodiments, during the formation of the absorbent core 50, the pocket 194 transition surface 198 connecting the bottom surface 196 of the pocket 194 to the first section 192 can be perpendicular to the planar surface of the first section 192. This can provide for an absorbent core 50 having an abrupt transition between the primary absorbent region 60 and the secondary absorbent region 62. The topsheet layer facing surface 52 can be profiled due to the presence of the primary absorbent region 60 having a first height H1 which is greater than the secondary absorbent region 62 which has a second height H2. The backsheet layer facing surface 54 of the absorbent core 50 can be flat to not have any dip or ridge formed in the scarfed surface. Utilizing the airlaying method described herein to form the single layer absorbent core 50 having a primary absorbent region 60 and a secondary absorbent region 62 can result in each region, primary absorbent region 60 and secondary absorbent region 62, having the same density even though the heights between the two regions, primary absorbent region 60 and secondary absorbent region 62, are different.

While an airlaying method has been described herein to form the absorbent core 50, the absorbent core 50 can be formed by employing various conventional methods and techniques provided that the absorbent core 50 has a primary absorbent region 60 having a first height H1 and a secondary absorbent region 62 having a second height H2 wherein the first height H1 is greater than the second height H2 and the density of the primary absorbent region 60 is the same as the density of the secondary absorbent region 62. For example, the absorbent core 50 can be formed by techniques such as, but not limited to, a dry-forming technique, a wet forming technique, a foam forming technique, or the like, as well as combinations thereof. Methods and apparatus for carrying out such techniques are well known in the art.

In various embodiments, the absorbent core 50 can have a tertiary absorbent region 64. The tertiary absorbent region 64 can facilitate folding of the absorbent article 10 in various regions to accommodate the body of the wearer and provide close to body fit of the absorbent article 10 to the body of the wearer. Such a tertiary absorbent region 64, while facilitating folding of various regions of the absorbent article 10 can prevent folding of various other regions of the absorbent article 10. Additionally, the tertiary absorbent region 64 can funnel body exudates emanating from the body of the wearer of the absorbent article 10 towards a desired location in the absorbent article 10 for storage.

The tertiary absorbent region 64 can be formed via an embossing process wherein raised elements are used to impart the desired embossing pattern to create a compression, an embossment, in the layers of the absorbent article 10, such as, for example, the absorbent core 50 and, in various embodiments, the topsheet layer 40 in addition to the absorbent core 50. For instance, a suitable process may include using thermal bonding wherein the absorbent article 10 is passed between two rolls (e.g., steel, rubber, etc.) where one is engraved with an embossing pattern and the other is flat. One or both rolls may be heated. In addition, an ultrasonic bonding technique may be employed to create the tertiary absorbent region 64.

In various embodiments, a tertiary absorbent region 64 can be formed by embossing a line of discrete dashes or dots in the absorbent core 50 and topsheet layer 40 or by embossing a continuous channel into the absorbent core 50 and topsheet layer 40. In various embodiments, the absorbent core 50 can be provided with multiple tertiary absorbent regions 64 which can be formed of lines of discrete dashes or dots, continuous channels, or combinations thereof. The tertiary absorbent region 64 can be formed in any suitable pattern to create an aesthetically pleasing surface as well as to perform various of the functions described above.

The size (i.e., length and width) of the embossment that defines the tertiary absorbent region 64 can be varied to alter the characteristics (i.e., resistance to folding, funnel capabilities) and appearance of the tertiary absorbent region 64. The spacing between the individual dashes and dots within a line of dashes and dots can also be varied for the same reasons. An embossment can be provided with any shape and configuration as deemed suitable. For example, an embossment can be in the shape of a circle, oval, square, rectangle, diamond, or any other geometric shape deemed suitable. An embossment can have any length in the longitudinal direction (X) as deemed suitable and a width in the transverse direction (Y) as deemed suitable.

In various embodiments, an absorbent core 50 can have a single tertiary absorbent region 64. In various embodiments, an absorbent core 50 can have two separate and distinct tertiary absorbent regions 64. In various embodiments, an absorbent core 50 can have more than two separate and distinct tertiary absorbent regions 64.

In various embodiments, a tertiary absorbent region 64 can be symmetric about the longitudinal axis 12 of the absorbent article 10. In various embodiments, a tertiary absorbent region 64 is not symmetric about the longitudinal axis 12 of the absorbent article 10. In various embodiments, a tertiary absorbent region 64 is symmetric about the transverse axis 14 of the absorbent article 10. In various embodiments, a tertiary absorbent region 64 is not symmetric about the transverse axis 14 of the absorbent article 10.

In various embodiments, a portion of a tertiary absorbent region 64 is oriented in a direction generally parallel to the longitudinal axis 12 of the absorbent article 10. In various embodiments, a portion of a tertiary absorbent region 64 is in an overlapping configuration with the longitudinal axis 12 of the absorbent article 10.

At least a portion of the tertiary absorbent region 64 of the absorbent core 50 can be located within the crotch region 24 of the absorbent article 10. In various embodiments, the tertiary absorbent region 64 can have a first portion located within the crotch region 24 of the absorbent article 10 and can have a second portion located within either the anterior region 20 or the posterior region 22 of the absorbent article 10. In various embodiments, the tertiary absorbent region 64 can have a first portion located within the crotch region 24, a second portion located within the anterior region 20, and a third portion located within the posterior region 22 of the absorbent article 10. In various embodiments, the tertiary absorbent region 64 can traverse the transverse axis 14 of the absorbent article 10 whereby portions of the tertiary absorbent region 64 are located on opposing sides of the transverse axis 14 of the absorbent article 10. In various embodiments, the tertiary absorbent region 64 can traverse the longitudinal axis 12 of the absorbent article 10 whereby portions of the tertiary absorbent region 64 are located on opposing sides of the longitudinal axis 12 of the absorbent article 10. In various embodiments, the tertiary absorbent region 64 traverses each of the longitudinal axis 12 and the transverse axis 14 of the absorbent article 10.

In various embodiments, a portion of a tertiary absorbent region 64 is adjacent to a portion of a primary absorbent region 60 of the absorbent core 50. In various embodiments, a first portion of a tertiary absorbent region 64 is adjacent to a first portion of a primary absorbent region 60 and a second portion of the same tertiary absorbent region 64 is adjacent to a second portion of the same primary absorbent region 60 of the absorbent core 50. In various embodiments, a tertiary absorbent region 64 can fully surround a primary absorbent region 60 and a portion of the tertiary absorbent region 64 can be adjacent to the primary absorbent region 60. In various embodiments, a tertiary absorbent region 64 can fully surround and be fully adjacent to a primary absorbent region 60. In various embodiments, the absorbent core 50 can have two primary absorbent regions 60 and a tertiary absorbent region 64 can be positioned between the two primary absorbent regions 60 to separate the two primary absorbent regions 60. In various embodiments, an absorbent core 50 can have two primary absorbent regions 60 and a tertiary absorbent region 64 can fully surround and be fully adjacent to each of the primary absorbent regions 60.

As the tertiary absorbent region 64 is formed by subjecting the absorbent core 50 to an embossing process, the absorbent material forming the tertiary absorbent region 64 is the same as the absorbent material forming each of the primary absorbent region 60 and the secondary absorbent region 62. As the tertiary absorbent region 64 is formed by an embossing process, the tertiary absorbent region 64 can have a third height H3 which is smaller than the first height H1 of the primary absorbent region 60 and which is smaller than the second height H2 of the secondary absorbent region 62. In various embodiments, the third height H3 of the tertiary absorbent region 64 is less than about 1, 2, 3, or 4 mm. Due to the embossing process, the tertiary absorbent region 64 will have a density which is greater than the density of each of the primary absorbent region 60 and the secondary absorbent region 62.

Backsheet Layer:

The backsheet layer 44 is generally liquid impermeable and is the portion of the absorbent article 10 which faces the garments of the wearer. The backsheet layer 44 can permit the passage of air or vapor out of the absorbent article 10 while still blocking the passage of liquids. Any liquid impermeable material may generally be utilized to form the backsheet layer 44. The liquid impermeable layer 44 can be composed of a single layer or multiple layers, and these one or more layers can themselves comprise similar or different materials. Suitable material that may be utilized can be a microporous polymeric film, such as a polyolefin film or polyethylene or polypropylene, nonwovens, and nonwoven laminates, and film/nonwoven laminates. The particular structure and composition of the backsheet layer 44 can be selected from various known films and/or fabrics with the particular material being selected as appropriate to provide the desired level of liquid barrier, strength, abrasion resistance, tactile properties, aesthetics, and so forth. In various embodiments, a polyethylene film can be utilized that can have a thickness in the range of from about 0.2 or 0.5 mils to about 3.0 or 5.0 mils. An example of a backsheet layer 44 can be a polyethylene film such as that obtainable from Pliant Corp., Schaumburg, Ill., USA. Another example can include calcium carbonate-filled polypropylene film. In still another embodiment, the backsheet layer 44 can be a hydrophobic nonwoven material with water barrier properties such as a nonwoven laminate, an example of which can be a spunbond, meltblown, meltblown, spunbond, four-layered laminate.

The backsheet layer 44 can, therefore, be of a single or multiple layer construction, such as of multiple film layers or laminates of film and nonwoven fibrous layers. Suitable backsheet layers 44 can be constructed from materials such as those described in U.S. Pat. No. 4,578,069 to Whitehead, et al., U.S. Pat. No. 4,376,799 to Tusim, et al., U.S. Pat. No. 5,695,849 to Shawver, et al., U.S. Pat. No. 6,075,179 to McCormack, et al., and U.S. Pat. No. 6,376,095 to Cheung, et al., each of which are hereby incorporated by reference thereto in its entirety.

Distribution Layer:

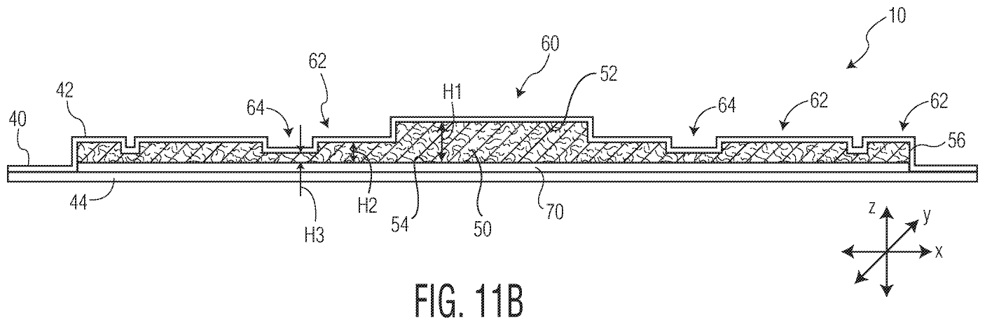

Referring to FIGS. 11A and 11B, which are cross-sectional views of an embodiment of the absorbent article 10 of FIG. 1 taken along lines 11A-11A and 11B-11B, in various embodiments, the absorbent article 10 can have a distribution layer 70 positioned below the absorbent core 50 in the depth direction (Z) of the absorbent article 10 such that the distribution layer 70 is between the absorbent core 50 and the backsheet layer 44. The distribution layer 70 can increase absorbency of the absorbent article 10. The distribution layer 70 can be constructed of various materials such as, but not limited to, hydroentangled webs, through air bonded carded webs, cellulosic fluff based materials, meltblown webs, and meltblown microfiber webs. The distribution layer 70 can include a hydrophilic material. In various embodiments, the distribution layer 70 can have a topographical texture such as, for example, a corrugation pattern.

In various embodiments, the distribution layer 70 can have a density of greater than about 0.1 grams per cubic centimeter. The density can be calculated utilizing the formula: density=basis weight (gsm)/thickness (mm)/1000. In various embodiments, the distribution layer 70 can have a basis weight from about 10, 20, 25, 30 or 50 gsm to about 60, 70, 80, 90, 100, 120, 140, 150, 160, 180 or 200 gsm.

In various embodiments, the distribution layer 70 can be a hydroentangled web. The hydroentangled web can include a hydroentangled spunbond material and a pulp material. The hydroentangled spunbond material can include a polypropylene material. The spunbond material can be present in an amount from about 10% or 15% to about 20% or 25% of the hydroentangled web. The pulp material can be present in an amount from about 75% or 80% to about 85%, 90% or 100% of the hydroentangled web. The hydroentangled web can have a basis weight from about 30 or 60 gsm to about 90, 200, or 300 gsm. Without being bound by theory, it is believed that a higher basis weight hydroentangled web can improve the absorbency of the distribution layer 70. It is further believed that an improved absorbency of the distribution layer 70 can further result in an improved fluid retention capacity of the absorbent article 10. The basis weight of the hydroentangled web can be balanced with the desired flexibility of the absorbent article 10. In various embodiments, the distribution layer 70 can be a pulp sheet material. In such embodiments, the distribution layer 70 can contain 100% pulp material. In such embodiments, the distribution layer 70 can have a basis weight from about 30 or 60 gsm to about 90, 200 or 300 gsm. In various embodiments, the distribution layer 70 can include a bicomponent fluid distribution layer, which can increase absorbency by providing a high void space and may be made of a through air bonded carded web, having a basis weight, in an embodiment, of between about 25 gsm and 100 gsm. In various embodiments, the distribution layer 70 can be constructed of a superabsorbent polymer-containing compressed sheet. In such embodiments, the superabsorbent polymer-containing compressed sheet can be a cellulosic fluff based material that can be a combination of cellulosic pulp and SAP enclosed with a tissue carrier and having a basis weight from about 40 to about 400 gsm. In various embodiments, the distribution layer 70 can be a meltblown microfiber web of polypropylene material and can have a basis weight from about 10 or 20 gsm to about 30, 50 or 100 gsm. In various embodiments, the meltblown microfiber web can be treated with wetting agents for adequate handling of body exudates. Examples of wetting agents can include, but are not limited to, surface active agents (or surfactants) having a hydrophilic lipophilic balance (HLB) of at least 6, 7 or 18. A variety of surfactants can be used and can include, but are not limited to, anionic, cationic, or neutral from a charge standpoint. Mixtures of surfactants and other wetting agents can also be used. A wetting agent add-on can range from about 0.1 or 0.2% to about 5 or 10%. In various embodiments, an add-on amount can be higher than 10%. For example, the meltblown microfiber web can be treated to impart hydrophilcity by either Aerosol GPG of Cytec or Ahcovel Base N-62 for example. Such material is available from Yuhan-Kimberly Ltd., Seoul, Korea and FlberTex, Malaysia.

The distribution layer 70 can be provided in any shape as deemed suitable for the absorbent article 10 such as, but not limited to, oblong, oval, rectangular, tear-dropped, hourglass, and racetrack. In various embodiments, the shape of the distribution layer 70 can have a shape which provides symmetry about at least one axis, longitudinal 12 and/or transverse 14, of the absorbent article 10. In various embodiments, the shape of the distribution layer 70 can be one in which there is no symmetry of the distribution layer 70 about either of the axes, longitudinal 12 or transverse 14 of the absorbent article 10.

Wings:

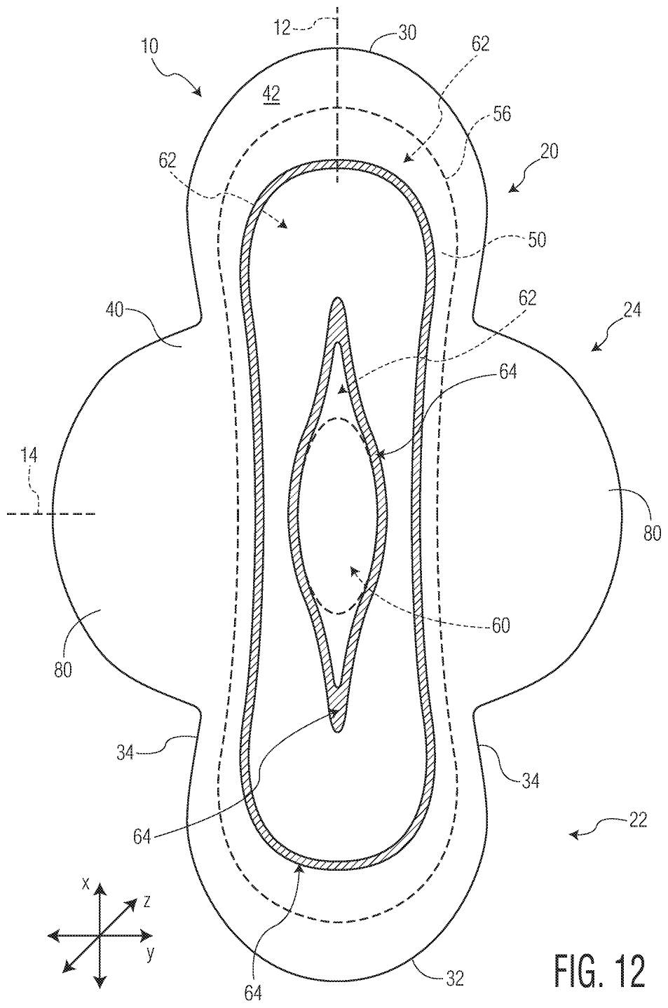

Referring to FIG. 12 which is an embodiment of an absorbent article 10, in various embodiments, the absorbent article 10 can have a pair of wings 80 extending outwardly, in the transverse direction (Y), from the absorbent article 10. The wings 80 can drape over the edges of the wearer's undergarment so that the wings 80 are disposed between the edges of the wearer's undergarment and her thighs. The wings 80 can serve at least two purposes. First, the wings 80 can prevent soiling of the wearer's undergarment by forming a barrier along the edges of the undergarment. Second, the wings 80 can be provided with fastener, such as, for example, a garment attachment adhesive or a hook, to keep the absorbent article 10 securely and properly positioned in the undergarment. The wings 80 can wrap around the crotch region of the wearer's undergarment to aid in securing the absorbent article 10 to the wearer's undergarment when in use. Each wing 80 can fold under the crotch region of the wearer's undergarment and the fastener can either form a secure attachment to the opposite wing 80 or directly to the surface of the wearer's undergarment. In various embodiments, the wings 80 can be an extension of materials forming the topsheet layer 40 and/or the backsheet layer 44 and can be bonded together along the sealed peripheral region. Such wings 80 can be integrally formed with the main portion of the absorbent article 10. In various embodiments, the wings 80 can be constructed of materials similar to the topsheet layer 40, the backsheet layer 44, or combinations of these materials. In various embodiments, the wings 80 can be separate elements bonded to the main body of the absorbent article 10. Examples of processes for manufacturing absorbent articles 10 and wings 80 include, but are not limited to, those described in U.S. Pat. No. 4,059,114 to Richards, U.S. Pat. No. 4,862,574 to Hassim, et al., U.S. Pat. No. 5,342,647 to Heindel, et al., U.S. Pat. No. 7,070,672 to Alcantara, et al., U.S. Publication No. 2004/0040650 to Venturino, et al., and international publication WO1997/040804 to Emenaker, et al., each of which are hereby incorporated by reference thereto in its entirety. It is to be understood that the wings 80 are optional and, in various embodiments, an absorbent article 10 can be configured without wings 80.

Example Absorbent Articles:

Referring to FIGS. 1, 2A, and 2B, an absorbent article 10 is exemplified in the form of a feminine hygiene product such as a menstrual pad or sanitary napkin. FIG. 1 provides a top down view of the exemplary embodiment of the absorbent article 10, FIG. 2A provides an exemplary embodiment of a cross-sectional view of the absorbent article 10 of FIG. 1 taken along line 2A-2A, and FIG. 2B provides an exemplary embodiment of a cross-sectional view of the absorbent article 10 of FIG. 1 taken along line 2B-2B. The absorbent core 50 of the absorbent article 10 can have a single primary absorbent region 60. The primary absorbent region 60 crosses over each of the longitudinal axis 12 and the transverse axis 14 of the absorbent article 10. In the longitudinal direction (X) of the absorbent article 10, portions of the primary absorbent region 60 are adjacent to portions of secondary absorbent regions 62. The absorbent article 10 has two tertiary absorbent regions 64 wherein one of the tertiary absorbent regions 64 is generally in the shape of a diamond and is oriented in the longitudinal direction (X) of the absorbent article 10. This diamond-like shaped tertiary absorbent region 64 has at two portions which are adjacent to two portions of the primary absorbent region 60 of the absorbent core 50. At least a portion of this tertiary absorbent region 64 is in an overlapping configuration with the longitudinal axis 12 of the absorbent article 10. The second tertiary absorbent region 64 is generally in the shape of a racetrack and is located closer to the perimeter 56 of the absorbent core 50 than the first tertiary absorbent region 64. The primary absorbent region 60 has a first height H1 greater than the second height H2 of the secondary absorbent regions 62 and greater than the third height H3 of the tertiary absorbent regions 64. The density of the tertiary absorbent regions 64 is greater than the density of either of the primary absorbent region 60 or the secondary absorbent regions 62.

FIGS. 11A and 11B, provide an exemplary illustration of an alternate cross-sectional view of the absorbent article 10 of FIG. 1 taken along lines 11A-11A and 11B-11B, respectively. The absorbent article 10 illustrated in FIGS. 11A and 11B further has a distribution layer 70 positioned between the absorbent core 50 and the backsheet layer 44 of the absorbent article 10.

FIG. 12 provides an exemplary illustration of an alternate embodiment of the absorbent article 10 of FIG. 1. The absorbent article 10 illustrated in FIG. 12 further has a pair of wings 80 extending in the transverse direction (Y).

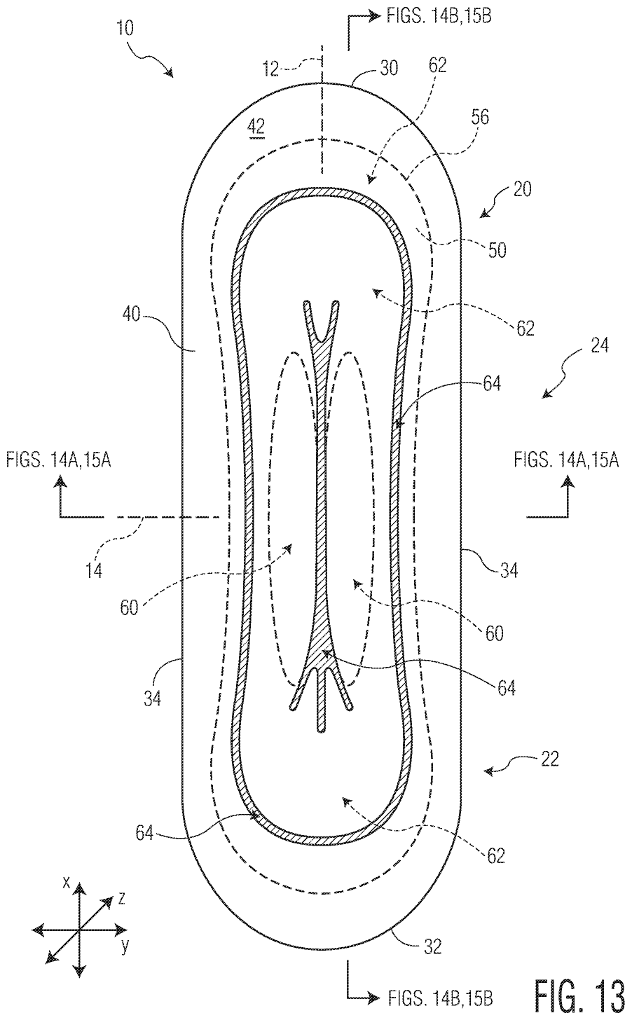

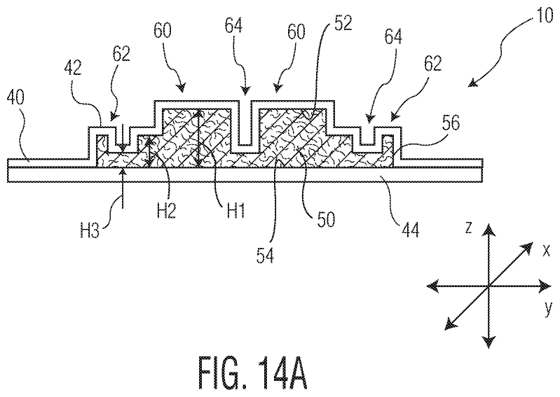

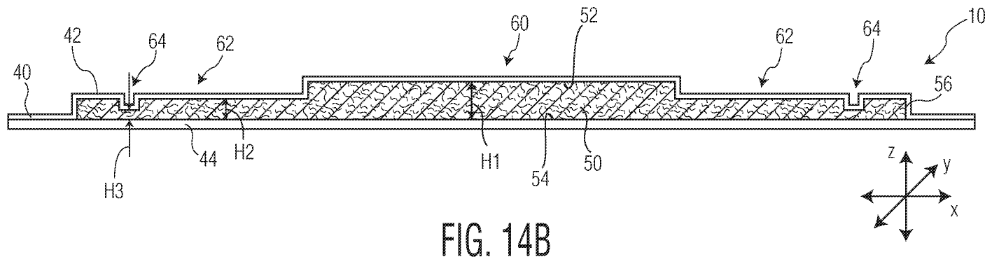

Referring to FIGS. 13, 14A, and 14B, an absorbent article 10 is exemplified in the form of a feminine hygiene product such as a menstrual pad or sanitary napkin. FIG. 13 provides a top down view of the exemplary embodiment of the absorbent article 10, FIG. 14A provides an exemplary embodiment of a cross-sectional view of the absorbent article 10 of FIG. 13 taken along line 14A-14A, and FIG. 14B provides an exemplary embodiment of a cross-sectional view of the absorbent article 10 of FIG. 13 taken along line 14B-14B. The absorbent core 50 of the absorbent article 10 can have two primary absorbent regions 60. Each of the primary absorbent regions 60 cross over the transverse axis 14 of the absorbent article 10, but neither of the primary absorbent regions 60 cross over the longitudinal axis 12 of the absorbent article 10. The absorbent article 10 has two tertiary absorbent regions 64 wherein one of the tertiary absorbent regions 64 is generally oriented in the longitudinal direction (X) of the absorbent article 10, is positioned between and separating the two primary absorbent regions 60, has a portion which is adjacent to each of the primary absorbent regions 60, and at least a portion of the tertiary absorbent region 64 is in an overlapping configuration with the longitudinal axis 12 of the absorbent article 10. The second tertiary absorbent region 64 is generally in the shape of a racetrack and is located closer to the perimeter 56 of the absorbent core 50 than the first tertiary absorbent region 64. The primary absorbent region 60 has a first height H1 greater than the second height H2 of the secondary absorbent regions 62 and greater than the third height H3 of the tertiary absorbent regions 64. The density of the tertiary absorbent regions 64 is greater than the density of either of the primary absorbent region 60 or the secondary absorbent regions 62.

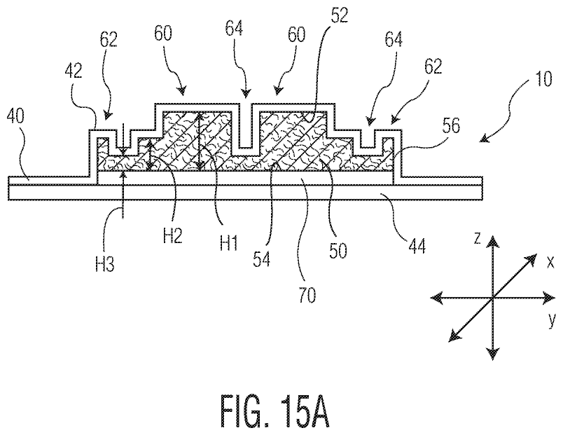

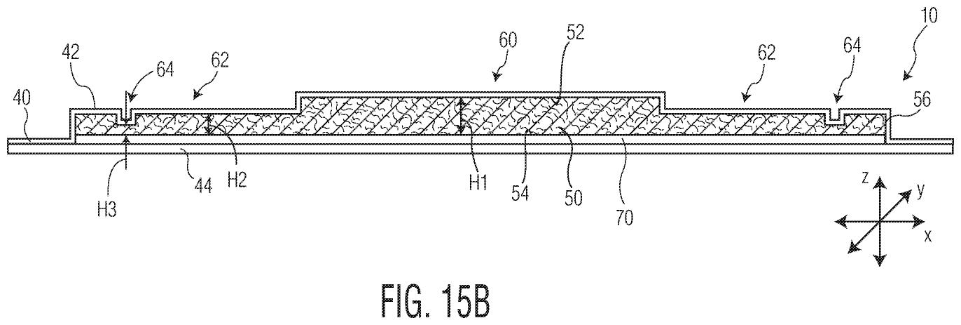

FIGS. 15A and 15B, provide an exemplary illustration of an alternate cross-sectional view of the absorbent article 10 of FIG. 13 taken along lines 15A-15A and 15B-15B, respectively. The absorbent article 10 illustrated in FIGS. 15A and 15B further has a distribution layer 70 positioned between the absorbent core 50 and the backsheet layer 44 of the absorbent article 10.

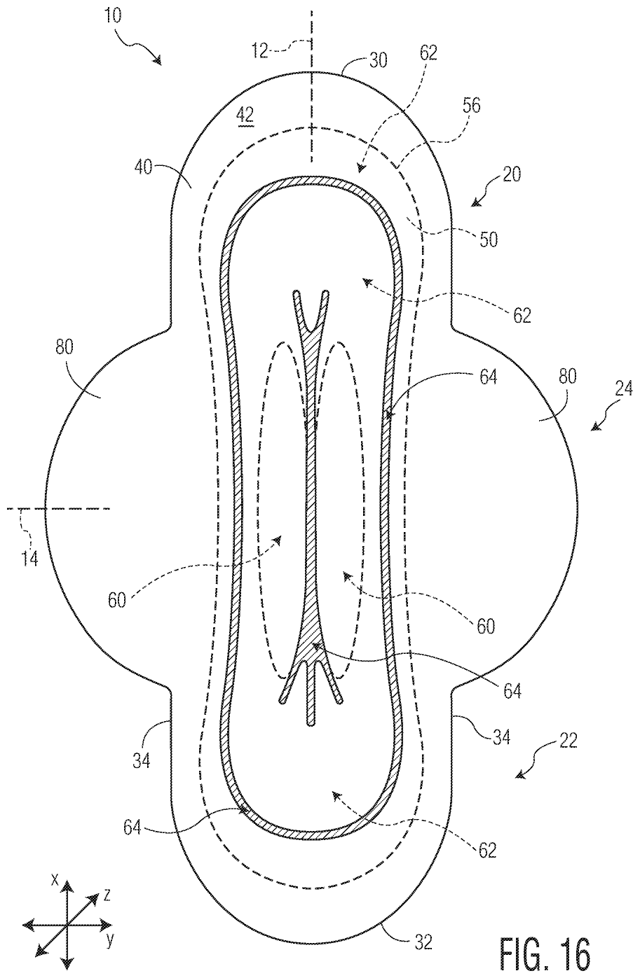

FIG. 16 provides an exemplary illustration of an alternate embodiment of the absorbent article 10 of FIG. 13. The absorbent article 10 illustrated in FIG. 16 further has a pair of wings 80 extending in the transverse direction (Y).

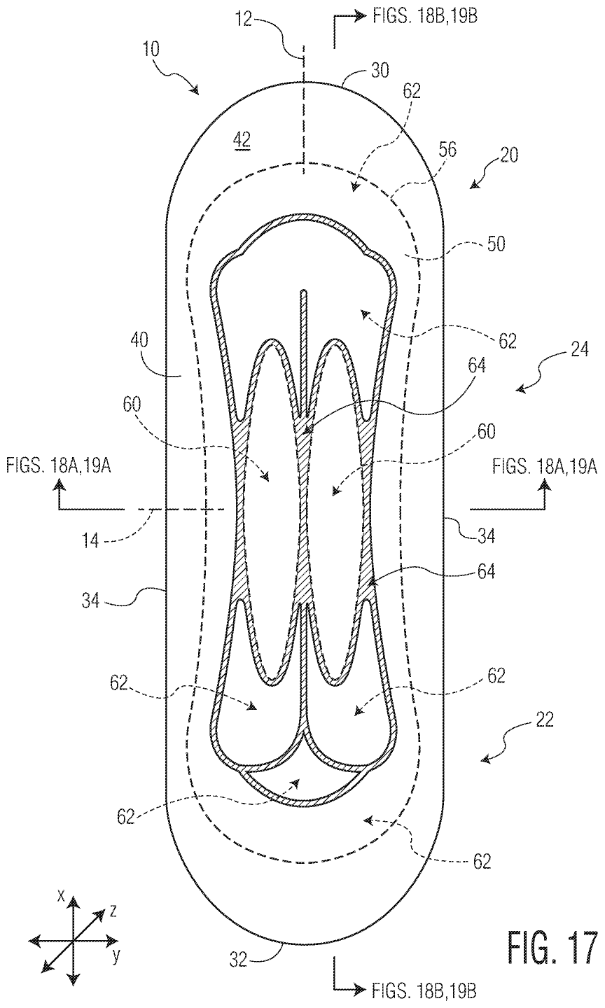

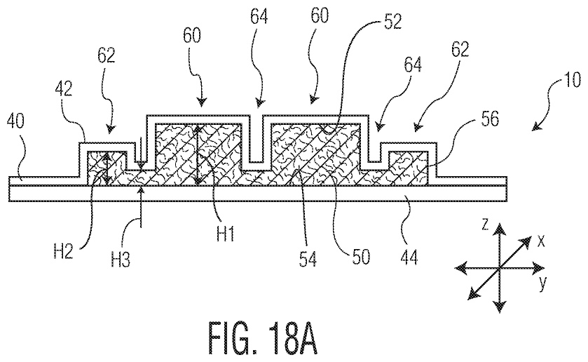

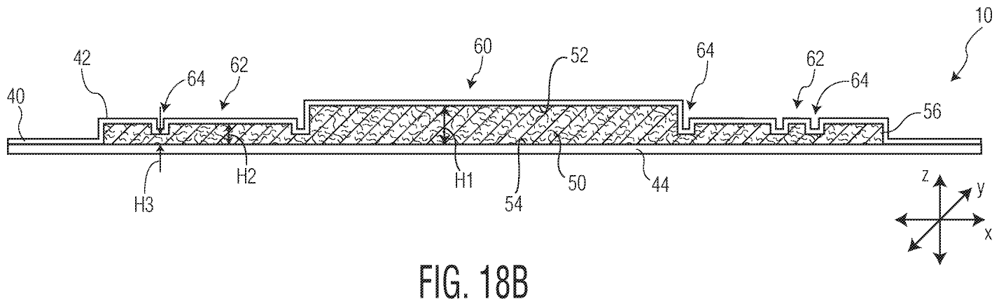

Referring to FIGS. 17, 18A, and 18B, an absorbent article 10 is exemplified in the form of a feminine hygiene product such as a menstrual pad or sanitary napkin. FIG. 17 provides a top down view of the exemplary embodiment of the absorbent article 10, FIG. 18A provides an exemplary embodiment of a cross-sectional view of the absorbent article 10 of FIG. 17 taken along line 18A-18A, and FIG. 18B provides an exemplary embodiment of a cross-sectional view of the absorbent article 10 of FIG. 17 taken along line 18B-18B. The absorbent core 50 of the absorbent article 10 can have two primary absorbent regions 60. Each of the primary absorbent regions 60 cross over the transverse axis 14 of the absorbent article 10, but neither of the primary absorbent regions 60 cross over the longitudinal axis 12 of the absorbent article 10. The absorbent article 10 has a single tertiary absorbent region 64 wherein the tertiary absorbent region 64 fully surrounds and is adjacent to each of the primary absorbent regions 60. The tertiary absorbent region 64 further surrounds and is adjacent to multiple smaller segments of secondary absorbent regions 62. The primary absorbent region 60 has a first height H1 greater than the second height H2 of the secondary absorbent regions 62 and greater than the third height H3 of the tertiary absorbent regions 64. The density of the tertiary absorbent regions 64 is greater than the density of either of the primary absorbent region 60 or the secondary absorbent regions 62.

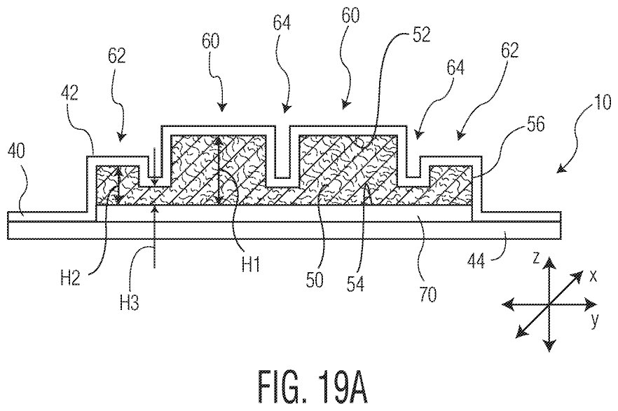

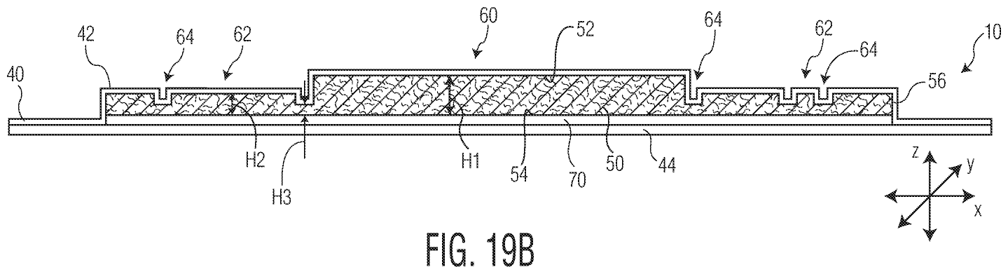

FIGS. 19A and 19B, provide an exemplary illustration of an alternate cross-sectional view of the absorbent article 10 of FIG. 17 taken along lines 19A-19A and 19B-19B, respectively. The absorbent article 10 illustrated in FIGS. 19A and 19B further has a distribution layer 70 positioned between the absorbent core 50 and the backsheet layer 44 of the absorbent article 10.

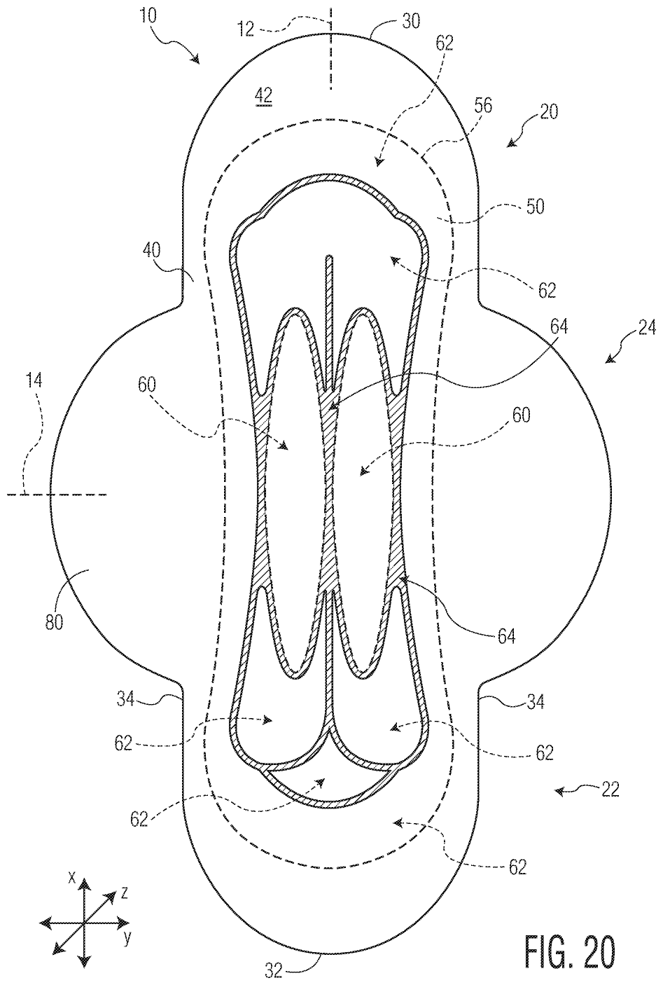

FIG. 20 provides an exemplary illustration of an alternate embodiment of the absorbent article 10 of FIG. 17. The absorbent article 10 illustrated in FIG. 20 further has a pair of wings 80 extending in the transverse direction (Y).

In the interests of brevity and conciseness, any ranges of values set forth in this disclosure contemplate all values within the range and are to be construed as support for claims reciting any sub-ranges having endpoints which are whole number values within the specified range in question. By way of hypothetical example, a disclosure of a range of from 1 to 5 shall be considered to support claims to any of the following ranges 1 to 5; 1 to 4; 1 to 3; 1 to 2; 2 to 5; 2 to 4; 2 to 3; 3 to 5; 3 to 4; and 4 to 5.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description are, in relevant part, incorporated herein by reference; the citation of any documents is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

What is claimed is:

1. An absorbent article comprising:
a. a topsheet layer;
b. a backsheet layer;
c. an absorbent core positioned between the topsheet layer and the backsheet layer, the absorbent core comprising:
i. a topsheet layer facing surface and a backsheet layer facing surface;
ii. a first primary absorbent region having a first height measured from the topsheet layer facing surface to the backsheet layer facing surface and a first density;
iii. a secondary absorbent region having a second height measured from the topsheet layer facing surface to the backsheet layer facing surface and a second density; and
iv. a tertiary absorbent region having a third height measured from the topsheet layer facing surface to the backsheet layer facing surface and a third density, wherein a portion of the tertiary absorbent region is adjacent to a portion of the primary absorbent region;
wherein the first height is greater than the second height, the second height is greater than the third height, the first density is the same as the second density, and the third density is greater than each of the first density and the second density.

2. The absorbent article of claim 1 wherein the tertiary absorbent region is formed by an embossment.

3. The absorbent article of claim 1 wherein a portion of the tertiary absorbent region extends in a longitudinal direction of the absorbent article.

4. The absorbent article of claim 1 wherein a portion of the tertiary absorbent region is in an overlapping configuration with a longitudinal axis of the absorbent article.

5. The absorbent article of claim 1 wherein the first primary absorbent region crosses over a longitudinal axis of the absorbent article.

6. The absorbent article of claim 1 further comprising a second primary absorbent region having a fourth height measured from the topsheet layer facing surface to the backsheet layer facing surface and a fourth density wherein the fourth height is the same as the first height and the fourth density is the same as each of the first density and the second density.

7. The absorbent article of claim 6 wherein a portion of the tertiary absorbent region is located between the first primary absorbent region and the second primary absorbent region of the absorbent article.

8. The absorbent article of claim 7 wherein the tertiary absorbent region is formed by an embossment.

9. The absorbent article of claim 8 wherein a portion of the tertiary absorbent region extends in a longitudinal direction of the absorbent article.

10. The absorbent article of claim 9 wherein a portion of the tertiary absorbent region is in an overlapping configuration with a longitudinal axis of the absorbent article.

11. The absorbent article of claim 6 wherein neither of the first primary absorbent region or the second primary absorbent region cross over a longitudinal axis of the absorbent article.

12. The absorbent article of claim 1 wherein the absorbent core comprises a cellulosic fluff material.

13. The absorbent article of claim 12 wherein the absorbent core further comprises a superabsorbent material.

14. The absorbent article of claim 1 further comprising a distribution layer.

15. The absorbent article of claim 14 wherein the distribution layer is positioned between the absorbent core and the backsheet layer of the absorbent article.
